(19) United States
(12) Patent Application Publication
(10) Pub. No.: US 2002/0026145 A1
(43) Pub. Date: Feb. 28, 2002

(54) Method and apparatus for emboli containment

(76) Inventors: Celso J. Bagaoisan, Union City, CA (US); Hung V. Ha, San Jose, CA (US); Mukund R. Patel, San Jose, CA (US); Gholam-Reza Zadno-Azizi, Newark, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/728,585

(22) Filed: Nov. 30, 2000

Related U.S. Application Data
(63) Continuation of application No. 08/813,023, filed on Mar. 6, 1997.

(51) Int. Cl. A61M 29/00; A61M 31/00; A61M 25/00

(52) U.S. Cl. 604/96.01; 604/97.01; 604/103.03; 604/527; 606/192

(57) ABSTRACT

Disclosed herein is a catheter for use in an emboli containment system. In one embodiment, the catheter has a tubular body with a metallic braid construction. Two lumens extend through the tubular body, the lumen being in a side-by-side configuration. One of the lumens functions as an inflation lumen, and is in fluid communication with an inflatable balloon mounted on the distal end of the catheter. The second lumen is adapted to receive other therapeutic catheters which comprise the emboli containment system. In another embodiment, apparatus is provided for treatment of a stenosis in a lumen in a blood-carrying vessel comprising a main catheter and a therapeutic catheter. The main catheter comprises a first flexible elongate tubular member having proximal and distal extremities. A sealing element is carried on the distal extremity of the first flexible elongate tubular member. The first flexible elongate tubular member has a main lumen therein extending from the proximal extremity to the distal extremity and exits through the distal extremity. An adapter is mounted on the proximal extremity of the first flexible elongate tubular member. The therapeutic catheter comprises a second flexible elongate tubular member having proximal and distal extremities. An element for performing work is mounted on the distal extremity of the second flexible elongate tubular member. An adapter is mounted on the proximal extremity of the second flexible elongate tubular member. The second flexible elongate tubular member is slidably mounted in the main lumen of the first elongate tubular member. A mechanism is carried by the first flexible elongate tubular member for causing the sealing element to form an occlusion in the vessel proximal of the stenosis to provide a working space distal of the sealing element. A mechanism is coupled to the attachment of the therapeutic catheter for causing operation of the element for performing work in the working space adjacent the stenosis.

### CROSS REFERENCE TO RELATED APPLICATIONS

This application is a continuation of application Ser. No. 08/813,023, filed on Mar. 6, 1997, which is a continuation-in-part of application Ser. No. 08/650,464 filed on May 20, 1996, the entirety of which is incorporated herein by reference, which is a continuation-in-part of application Ser. No. 08/464,579, filed Jun. 5, 1995, now U.S. Pat. No. 5,833,650.

### BACKGROUND OF THE INVENTION

The present invention generally relates to medical devices, and in particular, to catheters which can be used in an emboli containment system. This invention also relates to an apparatus and method for treating occluded vessels in living bodies and more particularly balloon catheters and balloon guide wires for treating occlusions in vessels in human bodies, as for example carotid arteries.

Balloon angioplasty, and other transluminal medical treatments, are well-known, and have been proven efficacious in the treatment of stenotic lesions in blood vessels. The application of such medical procedures to certain blood vessels, however, has been limited, due to the risks associated with creation of emboli during the procedure. For example, angioplasty is not the currently preferred treatment for lesions in the carotid artery, because of the possibility of dislodging plaque from the lesion, which can enter the various arterial vessels of the brain and cause permanent brain damage. Instead, surgical procedures are currently used, but these procedures present substantial risks.

One solution to this problem is the use of a multi-catheter emboli containment system, as disclosed in the above-referenced application Ser. No. 08/650,464. As disclosed therein, a treatment chamber within a blood vessel is formed by two occlusion balloons on opposite sides of a stenotic lesion, thereby preventing emboli migration during the treatment procedure. The chamber is created by two occlusion balloon catheters which are slidably disposed with respect to one another.

Emboli containment procedures of this type are advantageous, because they permit the clinician to utilize the benefits of transluminal treatment in a wider variety of blood vessels. However, the procedures require the complex coordination of multiple catheters. Consequently, it is desirable to have catheters which make it easier for the clinician to utilize an emboli containment system. It is also desirable that the catheters used in the emboli containment system have a high degree of flexibility, to navigate tortuous blood vessel networks.

Consequently, there exists a need for improved emboli containment catheters. This is especially true in the context of the “main” catheter, through which other catheters are inserted and controlled to form the emboli containment system. There is also a need for new and improved apparatus and methods which make it possible to treat occluded vessels without endangering the patient.

### SUMMARY OF THE INVENTION

The present invention advantageously provides as a main catheter an occlusive device adapted for use in a multi-catheter emboli containment system. In one aspect of the present invention, there is provided a catheter, comprising an elongate flexible tubular body having a proximal end and a distal end. The tubular body incorporates a metallic member, which may comprise a braid or a coil. A main lumen and an inflation lumen extend through the tubular body, and are in a side-by-side configuration. The main lumen is sized to receive a therapeutic and/or diagnostic device such as a balloon angioplasty catheter or an atherectomy catheter. The tubular body is provided with a manifold. The manifold has an aspiration port which is in fluid communication with the main lumen. The distal end of the tubular body also has a tip formed of a more flexible material than that used to form the tubular body.

In one preferred embodiment, an inflatable balloon is mounted on the distal end of the tubular body. An inflation port is also provided on the manifold in this embodiment. The inflation port is in fluid communication with the inflation lumen. In this embodiment, the inflatable balloon is formed of a block copolymer of styrene-ethylene-butylene-styrene.

In another preferred embodiment, the metallic braid or coil is formed of a metal selected from the group consisting of 304, 316, or 400 series stainless steel, nitinol, platinum, gold, Elgiloy (TM), or combinations thereof. Where a metallic braid is used, it may optionally have a braid density at a first point on the tubular body that is greater than the braid density of the metallic braid at a second point on the tubular body by at least 20 picks per inch. Similarly, where a metallic coil is used, it may optionally have a coil density at a first point on the tubular body that is greater than the coil density at a second point on the tubular body.

In another aspect of the present invention, there is provided a catheter comprising an elongate flexible tubular body having a proximal end and a distal end. Alternatively, there may be provided a circular cross-sectional configuration at the proximal end which is continuous with a distal end having a reduced internal and outer tubular body diameters. A first and second lumen extend through the tubular body from the proximal end to the distal end in a side-by-side configuration. The first lumen has a generally circular cross-sectional configuration at the proximal end and a generally oval cross-sectional configuration at the distal end. The second lumen has a diameter no smaller than 0.05 inches, preferably no smaller than 0.08 inches, and is adapted to slidably accommodate a therapeutic or diagnostic device.

In one preferred embodiment, an inflatable balloon is mounted on the distal end of the tubular body. The inflatable balloon is in fluid communication with the first lumen, such that fluid passing through the first lumen may be used to inflate or deflate the inflatable balloon. The second lumen size may vary in certain embodiments, such that in one embodiment, the second lumen has a diameter no smaller than about 0.05 inches, and is preferably no less than 0.080 inches.

In another aspect of the present invention, there is provided a catheter with variable stiffness, comprising a tubular body having a proximal end and a distal end. A metallic braid or metallic coil is within the tubular body. In one embodiment, the proximal end of the tubular body has a lower braid or coil density than the distal end. In another embodiment, the braid or coil density is kept constant along the length of the tubular body, and the tubular body is formed of materials with greater stiffness at the proximal end. In another embodiment, a combination of braids and coils of varying density can be used at various points along the tubular body, to create a catheter tubular body having a more flexible distal end.

In another aspect of the present invention, there is provided a method of making a catheter tubular body. The method comprises providing a first polymeric tube formed of a first material having a first melting point. The first polymeric tube is then inserted into a second polymeric tube to form a combined tube. The second polymeric tube is formed of a second material having a second melting point which is less than the first melting point. The combined tube is then placed adjacent to a third tube. The third tube is formed in part of the second material. The tubes are then heated to a temperature greater than the second melting point but less than the first melting point, such that the combined tube melt fuses with third tube to form a catheter tubular body having two lumen extending therethrough in a side-by-side configuration. The first material may be selected from the group comprising polyimide, polyamide, PET and PEEK, blends thereof and the second material may be selected from the group comprising Pebax (TM), polyethylene, nylon, or Hytrel (TM) or blends thereof. Preferably, the temperature of the heating step is from about 250° to 600° F. It is also preferred that the third tube incorporate a metallic member, such as a braid or coil.

In general, it is an objection of the present invention to provide an apparatus or an assembly and method which can be used with approved diagnostic and therapeutic devices while minimizing the opportunities for emboli to migrate downstream.

Another object of the present invention to provide an apparatus or assembly and method of the above character which makes it possible to perform therapeutic procedures without using perfusion.

Another object of the invention is to provide an apparatus or assembly and method of the above character in which the proximal balloon utilized is a balloon carried by a guide wire.

Another object of the invention is to provide an apparatus or assembly and method of the above characters in which the inflation fitting carried by the proximal extremity of the balloon-on-a-wire is removable so that catheters can be slid over the wire without removal of the wire from the site in which it is disposed.

Another object of the present invention is to provide an apparatus or assembly and method for treating occluded vessels of the above character which makes it possible to prevent downstream flow of debris or emboli.

Another object of the invention is to provide an apparatus and method which makes it possible to reverse the flow of blood in an occluded vessel during the time that a stenosis is being crossed.

Another object of the invention is to provide an apparatus and method of the above character in which a negative pressure is created within the vessel to reverse the flow of blood in the vessel.

Another object of the invention is to provide an apparatus and method of the above character in which it is only necessary to stop the flow of blood in a vessel of a patient for a very short period of time.

Another object of the invention is to provide an apparatus and method in which a working space is provided in the vessel free of blood for treatment of the stenosis.

Another object of the invention is to provide an apparatus and method of the above character in which material which is dislodged during the treatment of the occlusion or stenosis is removed by suction.

Another object of the invention is to provide an apparatus and method of the above character in which blood is shunted around the working space.

Another object of the invention is to provide an apparatus and method in which a cutting device is utilized for treatment of the stenosis or atheroma in the vessel and in which the material removed from the stenosis or atheroma is aspirated out of the operating space.

Another object of the invention is to provide an apparatus and method of the above character in which the amount of material removed from the stenosis or atheroma can be precisely controlled.

Another object of the invention is to provide an apparatus and method of the above character which makes it possible to treat stenoses or occlusion in the vessel which are normally not accessible for surgical procedures.

Another object of the invention is to provide an apparatus and method of the above character which utilizes two spaced apart balloons to create the working space in the vessel.

Another object of the invention is to provide an apparatus and method of the above character that can be utilized to create a working space in a vessel having a bifurcation therein and in which the working space includes the bifurcation.

Another object of the invention is to provide an apparatus and method of the above character which utilizes three spaced apart balloons to create the working space in the vessel having a bifurcation therein.

Another object of the invention is to provide an apparatus and method of the above character which includes a control console for controlling the inflation of the blood flow pump.

Another object of the invention is to provide an apparatus and method of the above character which is particularly adapted for use with the carotid vessels.

### BRIEF DESCRIPTION OF THE DRAWINGS

FIG. 1 is a side view of an embodiment of the catheter of the present invention.

FIG. 2 is a cross-sectional view of catheter of FIG. 1 along lines 2-2.

FIG. 3 is a cross-sectional view of the catheter of FIG. 1 along lines 3-3.

FIG. 4 is a longitudinal cross-sectional view of the distal end of the catheter of FIG. 1.

FIG. 5 is an enlargement of the region circumscribed by lines 5-5 of the catheter of FIG. 4.

FIG. 6 is an illustration of the catheter of the present invention as used in an emboli containment system.

FIG. 7 is a cross-sectional view of the emboli containment system of FIG. 6 along lines 7-7.

FIG. 8 is a cross-sectional view of the emboli containment system of FIG. 6 along lines 8-8.

FIG. 9 is a cross-sectional view of the emboli containment system of FIG. 6 along lines 9-9.

FIGS. 10A-E illustrate the use of an embodiment of the catheter of the present invention in an emboli containment treatment procedure.

FIG. 11 is a side-elevational view partially in section showing the catheter apparatus or assembly of the present invention for treating occluded vessels.

FIG. 12 is a cross-sectional view taken along the line 12-12 of FIG. 11.

FIG. 13 is a cross-sectional view taken along the line 13-13 of FIG. 11.

FIG. 14 is a cross-sectional view taken along the line 14-14 of FIG. 11.

FIG. 15 is a schematic illustration of how the catheter apparatus shown in FIG. 11 is deployed in a carotid artery.

FIGS. 16A-16E are illustrations showing the various steps utilized in deployment of the catheter apparatus in performing the method of the present invention in a vessel where a bifurcation is not present.

FIG. 17 is a side-elevational view partially in section of another embodiment of a catheter apparatus or assembly incorporating the present invention for treating occluded vessels using an atherectomy device.

FIG. 18 is a cross-sectional view taken along the line 18-18 of FIG. 17.

FIG. 19 is a cross-sectional view taken along the line 19-19 of FIG. 17.

FIG. 20 is a side-elevational view in section of the distal extremity of another embodiment of a catheter apparatus incorporating the present invention and utilized for delivering an expandable stent to a stenosis.

FIG. 21A is a schematic illustration showing the manner in which the apparatus of the present invention is utilized in connection with vessels of a patient in performing the method of the present invention.

FIG. 21B is an additional partial schematic illustration showing interconnections in the catheter apparatus shown in FIG. 21A.

FIG. 22 is a plan view of another embodiment of a catheter apparatus incorporating the present invention.

FIG. 23 is a cross-sectional view taken along the line 23-23 of FIG. 22.

FIG. 24 is an end elevational view looking down the line 24-24 of FIG. 22.

FIGS. 25A, B, C, and D are illustrations or cartoons showing the method of the present invention being utilized with the apparatus shown in FIG. 21 in a vessel having a bifurcation therein.

FIG. 26 is a side-elevational view of a main catheter incorporating the present invention.

FIGS. 26A and 26B are partial side-elevational views of the distal extremities showing alternative embodiments of the main catheter of the present invention incorporating, respectively, Judkins left shape and Judkins right shape in their distal extremities.

FIG. 27 is a cross-sectional view taken along the line 27-27 of FIG. 26.

FIG. 28 is a cross-sectional view taken along the line 28-28 of FIG. 26.

FIG. 29 is an enlarged partial cross-sectional view of the distal extremity of the catheter shown in FIG. 26.

FIG. 30 is a side-elevational view of the balloon-on-a-wire construction incorporating the present invention.

FIG. 31 is a cross-sectional view taken along the line 31-31 of FIG. 30.

FIG. 32 is an enlarged cross-sectional view of the distal extremity of the construction in FIG. 30.

FIG. 33 is a cross-sectional view similar to FIG. 32 but showing a different embodiment utilizing a twisted dual core.

FIG. 34 is a cross-sectional view similar to FIG. 32 but showing the use of a twisted core.

FIG. 35 is a cross-sectional view of the proximal removable fitting of the construction shown in FIG. 30.

FIG. 36 is a side-elevational view partially in cross section of an irrigation catheter incorporation the present invention.

FIGS. 36A and 36B are side-elevational views of the distal extremities of additional embodiments of irrigation catheters incorporating the present invention.

FIGS. 37-43 are cartoons showing the manner in which the apparatus of the present invention shown in FIGS. 26-36 is used performing a therapeutic procedure in accordance with the present invention.

FIG. 44 is a side-elevational view partially in cross-section of another embodiment of a main catheter incorporating the present invention.

FIG. 45 is a side-elevational view partially in cross-section showing another embodiment of an irrigation catheter incorporating the present invention.

FIGS. 46-50 are cartoons showing the manner in which a therapeutic carotid procedure is performed in accordance with the present invention where there is a bifurcation.

FIG. 51 is a side-elevational view partially in section of another embodiment of a balloon-on-a-wire incorporating the present invention.

FIG. 52 is a cross-sectional view taken along the line 52-52 of FIG. 51.

FIG. 53 is a side-elevational view in section of another embodiment of a catheter apparatus incorporating the present invention for treating occluded vessels.

FIG. 54 is a side-elevational view in section similar to FIG. 53 but showing the apparatus in FIG. 53 with the self-expandable sealing means deployed.

FIG. 55 is a side-elevational view in section of another embodiment of a catheter apparatus incorporating the present invention for treating occluded vessels.

FIG. 56 is a view similar to FIG. 55 but showing the self-expandable sealing means deployed.

### DETAILED DESCRIPTION OF THE PREFERRED EMBODIMENT

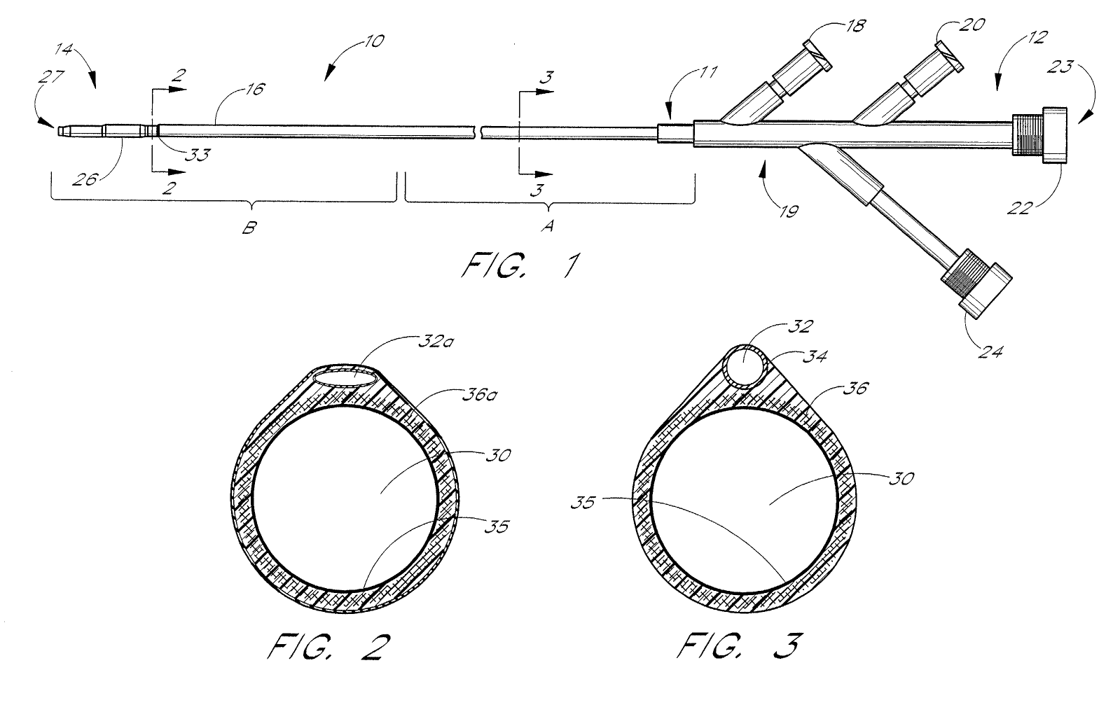

Referring to FIG. 1, there is depicted an embodiment of the balloon catheter of the present invention. Although illustrated and described below in the context of an emboli containment system featuring balloon dilatation treatment of a stenotic lesion, it is to be understood that the present invention can be easily adapted to a variety of emboli containment treatment applications. For example, the present inventors contemplate that the catheter of the present invention can be used in emboli containment treatment procedures which include atherectomy, stent implantation, drug delivery, as well as other applications. Furthermore, although depicted and described as a two lumen catheter, it should be appreciated that the present invention may also be adapted to catheters having more than two lumen. The manner of adapting the present invention to these various treatments and structures will become readily apparent to those of skill in the art in view of the description which follows.

Catheter 10 generally comprises an elongate flexible tubular body 16 extending between a proximal control end 12 and a distal functional end 14. Tubular body 16 has a main lumen 30 which extends between ends 12 and 14. Main lumen 30 terminates in a proximal opening 23 and a distal opening 27. A smaller inflation lumen 32, configured in a side-by-side relationship with main lumen 30, extends along the length of tubular body 16 and may terminate within or near an occlusion balloon 26 mounted on the distal end 14 of catheter 10, as described below. Inflation lumen 32 is in fluid communication with balloon 26, such that fluid passing through inflation lumen 32 may be used to inflate or deflate balloon 26. In some embodiments, the inflation lumen may originate at a point distal to the proximal end 12, and extend distally from that point in a side-by-side configuration with main lumen 30.

In some embodiments, instead of an occlusion balloon 26, distal end 14 is provided with a mechanical occlusive device such as a pull-wire activated braid which filters all particles larger than 12 microns. Alternatively, other occlusive filtering devices may also be used, as is known by those of skill in the art.

A control manifold 19 is provided at the proximal end 12 of catheter 10. Control manifold 19 is generally provided with a number of ports to provide access to the catheter lumen. For example, for the embodiment depicted in FIG. 1, control manifold 19 is provided with a catheter end-access port 22 and a catheter side-access port 24, to provide an introduction point for the insertion of other catheters into lumen 30. Ports 22 and 24 are preferably provided with standard Touhy Borst connectors, although other sealing type connectors, such as a hemostasis valve, may be used. Manifold 19 is also provided with an aspiration port 20 which is in fluid communication with lumen 30, for attachment of devices to aspirate fluid into opening 27, through lumen 30, and out port 20. An inflation port 18, in fluid communication with lumen 32, is further provided on manifold 18 for attachment of devices to inflate or deflate balloon 26. In one preferred embodiment, ports 18 and 20 are provided with standard luer connectors, to facilitate attachment of standard inflation or aspiration apparatus, respectively, to ports 18 and 20. Other embodiments of catheter 10 may feature more or less ports, depending upon the number of lumen in the catheter and the desired functionalities of the catheter.

Manifold 19 is preferably formed out of hard polymers or metals, which possess the requisite structural integrity to provide a functional access port to the catheter lumen, such as for balloon inflation or fluid aspiration. In one preferred embodiment, manifold 19 is integrally formed out of medical grade polycarbonate. Other suitable materials may be used to form manifold 19, such as polyvinyl chloride, acrylics, acrylonitrile butadiene styrene (ABS), nylon, and the like.

Manifold 19 is attached to tubular body 16 so that the various ports are placed in communication with the appropriate lumen, as described above in connection with FIG. 1. Preferably, a strain relieving connector 11 is used to join manifold 19 to tubular body 16. For the embodiment depicted in FIG. 1, strain relieving connector 11 consists of a length of flexible polymeric tubing, such as 40 durometer (D) Pebax (TM), or other polyether block amides, and other similar materials. Tubular body 16 is inserted in one end of strain relieving connector 11, and the other end of strain relieving connector 11 is inserted into manifold 19. Suitable adhesives, such as a cyanoacrylate, epoxies, or uv curable adhesives, may be used to bond manifold 19 to strain relieving connector 11. Alternately, manifold 19 may also be insert molded with the tubular body 16, as is known by those of skill in the art. Adhesives may also be used to bond the strain relieving connector 11 to tubular body 16, or alternately, conventional heat bonding, as known to those of skill in the art, may be used to attach tubular body 16 to strain relieving connector 11.

The length of tubular body 16 may be varied considerably depending upon the desired application. For example, where catheter 10 is to be used as part of an emboli containment system for treatment of carotid artery disease, with catheter 10 being introduced at the groin, the length of tubular body 16 may range from 80 to 110 centimeters, and is preferably 95 cm. Other treatment procedures, requiring a longer or shorter tubular body 16, are easily accommodated by the present invention, by forming a tubular body 16 of the desired length during the manufacturing process.

The outer diameter of tubular body 16 may also be varied considerably, and in most cases, will depend upon the intended treatment procedure for which catheter 10 will be used. That is, the outer diameter of tubular body 16 must be large enough to be capable of forming a main lumen 30 which can slidably accommodate the other catheters used in the emboli containment system, as described in detail below. However, the outer diameter of tubular body 16 must also be smaller than the internal diameter of smallest blood vessel through which catheter 10 passes during the selected treatment procedure. In general, the diameter of main lumen 30 may range from at least about 0.05 inches to about 0.12 inches, and be capable of accommodating many types of catheters to be used therein, while still maintaining a low profile for the diameter of tubular body 16.

For many treatment applications, it has been found that a tubular body having an outside diameter of no more than about 0.135 inches (10 French) is preferred. Advantageously, with an outer diameter of this size, main lumen 30 may have an internal diameter of about 0.10 inches, making lumen 30 capable of accommodating a wide variety of treatment catheters, or catheters used for diagnostic purposes. Of course, as will be appreciated by those of skill in the art, where the catheters intended to be inserted into lumen 30 are known to have outer diameters significantly smaller than 0.10 inches, such that lumen 30 may be smaller than 0.10 inches and still accommodate them, a tubular body 16 having an outer diameter of less than 0.135 inches may be selected.

Although not required, the interior surface of lumen 30 may be provided with a liner 35 formed of a lubricous material, to reduce the frictional forces between the lumen surface and the catheters which are inserted into lumen 30. In one preferred embodiment, liner 35 is formed out of polytetrafluoroethylene (PTFE). Lubricous materials other than PTFE, which are biocompatible, fairly flexible, and easily mounted to other polymeric materials of the type used to form catheter tubular bodies, may also be used to form liner 35. Examples of such materials include polyethylene, Pebax (TM), nylon, and the like. Where increased flexibility of the distal end 14 of catheter 10 is desired, Pebax (TM) may be used in place of PTFE along a selected portion of distal end 14, such as the distal most 15-20 cm of end 14.

To minimize the outer diameter of tubular body 16, it is preferable that inflation lumen 32 be as small as possible in accordance with its function. That is, inflation lumen 32 is preferably no larger than required to provide sufficient fluid to balloon 26 for rapid inflation, or so that fluid may be quickly withdrawn from balloon 26 during deflation. For compliant expansion balloons of the type described below, inflation lumen diameters of from about 0.008 inches to about 0.018 inches are satisfactory, with a diameter of about 0.014 inches being preferred for some applications.

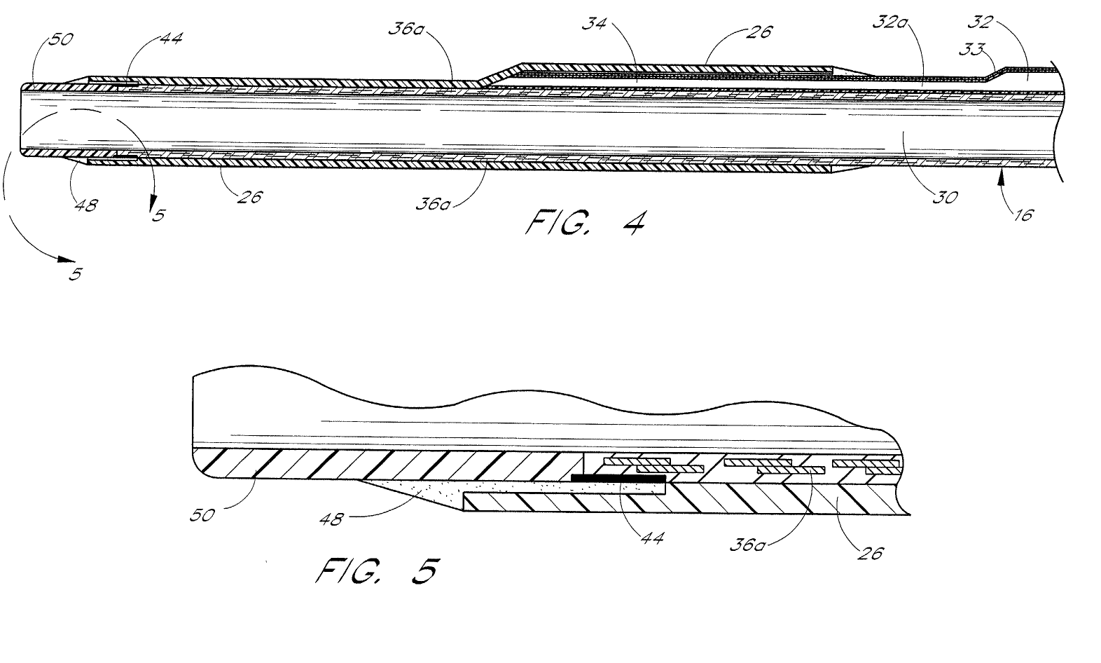

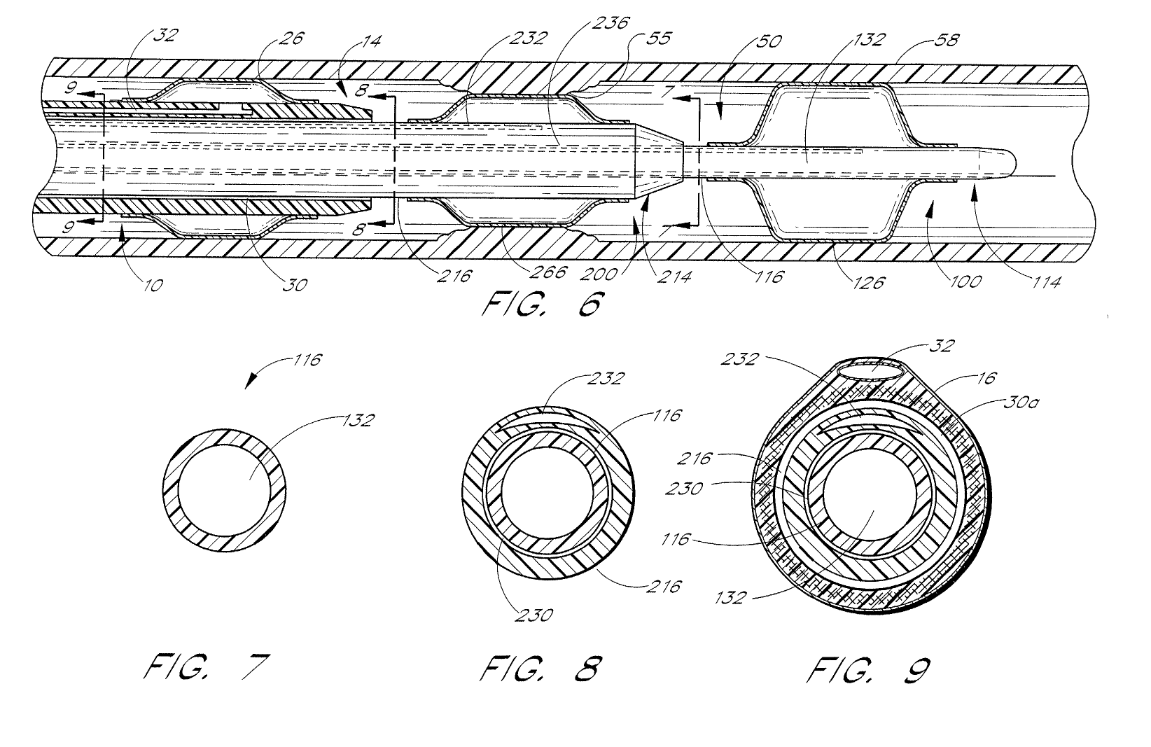

Furthermore, in one embodiment, as illustrated in FIGS. 1-3, the outer diameter of tubular body 16 just proximal to balloon 26 is minimized by providing an inflation lumen 32a with an oval cross-sectional configuration, as illustrated in FIG. 3. Preferably, inflation lumen 32a has an oval cross-sectional configuration which extends proximally from the proximal edge balloon 26 by a distance of at least 0.1 cm, more preferably 1 cm, and optimally by a distance equal to the length of tubular body 16. For ease of manufacturing, the cross-sectional configuration of lumen 32 at points further proximal to balloon 26 may be generally circular, as illustrated in FIG. 2. Where the lumen configuration differs from proximal to distal end, as illustrated in FIGS. 2 and 3, a region of transition 33 is provided wherein the lumen configuration changes from circular to oval.

It will be appreciated by those of skill in the art that other cross-sectional configurations of lumen 32a may be provided and still function to reduce the profile of tubular body 16. For example, triangular, rectangular, or other non-oval cross sectional configurations are easily adapted to lumen 32a, and the manner of incorporating such alternative cross-sectional configurations will be readily apparent to those of skill in the art in view of the description which follows.

A variety of different manufacturing methods may be used to alter the cross-sectional configuration of lumen 32, as will be appreciated by those of skill in the art. In one preferred method, lumen 32 is formed of a polymeric tube, such as a polyimide tube, which has been compressed at one end so that it has the desired oval shape. The polyimide tube is then inserted into a second tube formed of a material having a lower melting point than polyimide, such as 72D Pebax (TM). The combination is then heat bonded to another tube defining main lumen 30, such as a braided Pebax (TM) tube, as described below. The heat bonding takes place at a temperature greater than the melting temperature of Pebax (TM), but less than the melting temperature of polyimide, so that the Pebax (TM) tubes melt fuse to form the two lumen tubular body.

Alternately, the cross-sectional configuration, as well as the cross-sectional area of lumen 32, may also be altered by joining two separate polymeric tubes together to form a continuous inflation lumen 32. One of the tubes, corresponding to the proximal end of catheter 10 as shown in FIG. 3, may have a circular cross-sectional configuration. The second tube, corresponding to the distal end of catheter 10 as shown in FIG. 2, has an oval configuration. One end of a mandrel may be inserted into each of the tubes, and conventional heat bonding may be used to create the cross-sectional configuration transition. As before, the combined tube may then be heat bonded to a second tube defining main lumen 30 to form tubular body 16.

As illustrated in FIG. 1, an inflatable balloon 26 is mounted on the distal end 14 of catheter 10. In most applications where catheter 10 is to be used in an emboli containment treatment procedure, inflatable balloon 26 will function as an occlusion balloon, to prevent blood from passing through the blood vessel distal of balloon 26. Thus, inflatable balloon 26 is preferably able to expand to fit a variety of different blood vessel diameters. Accordingly, it is preferred that inflatable balloon 26 have a compliant expansion profile, tending to increase in radial diameter with increasing inflation pressure. To achieve this, balloon 26 may be made out of materials which impart such expansion characteristics, including elastomeric materials such as latex or silicone. In one preferred embodiment, inflatable balloon 26 is formed out of a material comprising a block copolymer of styrene-ethylene-butylene-styrene, sold under the trade name C-Flex (TM). Further details as to balloons of this type are disclosed in our copending application entitled PRE-STRETCHED CATHETER BALLOON, Ser. No. 08/812,139, filed Mar. 6, 1997, now abandoned, the entirety of which is incorporated by reference.

Inflatable balloon 26 may be placed in fluid communication with lumen 32a via a fill hole (not shown) extending through tubular body 16 within balloon 26, such that fluid may be introduced into lumen 32 through inflation port 18 to inflate balloon 26. Alternately, lumen 32a may terminate within balloon 26, to provide the requisite fluid communication. Balloon 26 may be attached to tubular body 16 by any suitable manner known to those of skill in the art, such as adhesives or heat bonding.

Tubular body 16 must have sufficient structural integrity, or “stiffness,” to permit catheter 10 to be advanced through vasculature to distal arterial locations without buckling or undesirable bending of tubular body 16. However, it is also desirable for tubular body 16 to be fairly flexible near distal end 14, so that tubular body 16 may be navigated through tortuous blood vessel networks. Thus, in one preferred embodiment, tubular body 16 is made to have variable stiffness along its length, with the proximal portion of tubular body 16 being less flexible than the distal portion of tubular body 16. Advantageously, a tubular body 16 of this construction enables a clinician to more easily insert tubular body 16 into blood vessel networks difficult to reach by a tubular bodies having uniform stiffness. This is because the stiffer proximal portion provides the requisite structural integrity needed to advance tubular body 16 without buckling, while the more flexible distal region is more easily advanced into and through tortuous blood vessel passageways.

In one preferred embodiment, variable stiffness along the length of tubular body 16 is achieved by forming a polymeric tubular body 16 which incorporates along its length a variable stiffness metallic member. The metallic member may comprise a braid or coil, and may have varying braid density or coil pitch at different points along the catheter tubular body. For example, as shown in FIGS. 2 and 3, tubular body 16 may be provided with a braid 36 incorporated into the wall structure of tubular body 16. Referring to FIG. 1, to achieve variable stiffness, proximal region A of catheter 10 is provided with a metallic braid 36 having a lower braid density than that present in the metallic braid 36a of distal region B. The lower braid density of proximal region A permits polymer flow in between the braids during the formation of the tubular body. Because the polymer is relatively stiffer than the braid, the lower braid density results in proximal region A being less flexible, or “stiffer”, than distal region B. In one preferred embodiment, the braid density of proximal region A varies from 60 to 80 picks per inch, while that of region B varies from 90 to 110 picks per inch.

As will be appreciated by those of skill in the art, metallic members other than braids may be incorporated into tubular body 16 to create variable stiffness. For example, a metallic coil may be introduced into tubular body 16. The coil may have different pitch along the length of tubular body 16, such that region A is provided with a coil having a lower pitch than that present in region B. The manner of adapting a coil, and other metallic members, to the catheter tubular body in place of a braid will become readily apparent to those of skill in the art in view of the description which follows.

The precise density of the braiding provided to regions A and B can be varied considerably at the point of manufacture, such that catheters having a variety of different flexibility profiles may be created. Moreover, the braid density may be varied within catheter regions A and B as well, by providing a metallic braid which has a braid density gradient along its length. For example, the most proximal part of region A may be provided with a metallic braid 36 having a braid density of about 60 picks per inch, with the braid density increasing distally at a certain rate so that the final pick count is not more than 110 picks per inch at the distal end.

A variety of different metals, known to be ductile and shapeable into fine wires and flat ribbons, having a diameter of about 0.0005 inches to about 0.005 inches for wires, or the same thickness for a ribbon, may be used to form the metallic braids 36 and 36a or metallic coils. For example, stainless steel, platinum, gold and nitinol, or combinations thereof are all suitable metals. In one preferred embodiment, braid 36 is formed of stainless steel, and has a braid density which varies from 70 picks per inch at the most proximal part of region A, to 100 picks per inch at the most distal part of region B.

Metallic braids 36 may be introduced into the structure of tubular body 16 through conventional catheter forming techniques. For example, tubular body 16 may be formed by braiding over a 72D Pebax (TM) tube that has a removable core mandrel in the internal diameter supporting the Pebax (TM) tube, and then inserting the braided tube into a 72D Pebax (TM) outer tube at the proximal region A and a 35D Pebax (TM) tube at the distal region B, so that the braid is sandwiched between the inner and outer tubes. A stainless steel support mandrel may be inserted into the removable core mandrel as additional support. A shaping container such as a fluorinated ethylene propylene (FEP) shrink tube is inserted over the outer Pebax (TM) tube, and the entire apparatus may then be placed in a hot box or oven kept at a temperature slightly greater than the melting temperature of the Pebax (TM) tubes. The Pebax (TM) tubes will melt and fuse together, and once cooled, will form a tubular body incorporating the metallic braid. The shaping container and mandrels may then be removed and discarded.

In another embodiment, variable stiffness of tubular body 16 may be achieved by forming regions A and B of tubular body 16 out of polymeric materials having differing degrees of stiffness. For example, one half of an inner tube of 72D Pebax (TM) may be inserted into an outer tube of 35D Pebax (TM), and the other half of the inner tube may be inserted into a 72D Pebax (TM) outer tube. The combination may then be heat fused, as described above. The 35D/72D Pebax (TM) combination forms a more flexible tubular body than the region 72D/72D Pebax combination. More or less flexible materials may be used as desired to alter the flexibility of the resulting tubular body. Furthermore, the flexibility of the various regions of a tubular body formed in this manner may be varied further by incorporating a metallic member having either a uniform density, or a varying density, into the tubular body, as described above.

In another preferred embodiment, variable stiffness along the length of the tubular body may be achieved by using different metallic members in regions A and B. For example, proximal region A may be provided with a multilayer coil, while distal region B may be provided with a braid. Alternately, proximal region A may be provided with a metallic braid, while distal region B may be provided with a single layer coil. As discussed above, the densities of the metallic members in the respective sections may be varied considerably to select for a desired variable stiffness profile, as will be appreciated by those of skill in the art.

In one preferred embodiment, variable stiffness along the length of the tubular body is achieved by keeping the braid density constant along the length of tubular body 16 and then forming the proximal and distal portions of tubular body 16 of polymeric materials of differing stiffness. For example, braid density may be uniform and range from 60-80 picks/inch, more preferably be about 70 picks/inch, with region A being formed of 72D Pebax (TM) and region B being formed of 25-50D Pebax (TM). Alternately, region A can be formed of high density polyethylene and region B of low density polyethylene.

Moreover, any of a variety of different polymeric materials known by those of skill in the art to be suitable for catheter body manufacture may be used to form tubular body 16. For example, tubular body 16 may be formed out of Pebax (TM), blends of Pebax (TM), and nylons, polyetheretherketone (PEEK), polyethylenes, and Hytrel (TM), and the like. Different materials might also be combined or blended to select for desirable flexibility properties.

Also, although tubular body 16 has been described in the context of having two regions of differing flexibility, it will be readily appreciated by those of skill in the art that three or more regions of differing flexibility may easily be provided, by adapting the teachings contained herein.

In the above-discussed embodiments, and all other embodiments of the present invention, it may be preferred to provide main lumen 30 and the outer surface of tubular body 16 with a hydrophillic coating, a hydrophobic coating, or combinations thereof. For example, main lumen 30 may be provided with a hydrophobic coating, such as silicone, while tubular body 16 is provided with a hydrophillic coating, such as polyvinyl pyrrolidone (PVP), polyurethane blends, copolymers of acrylonitrile, and the like. Other hydrophobic and hydrophillic coatings, as known to those of skill in the art, may also be used. In addition, any of a variety of antithrombogenic coatings, such as heparin, may also be applied to the catheter of the present invention, alone or in combination with other coating types.

Referring to FIGS. 4 and 5, there is illustrated a cross-sectional view of the distal end 14 of catheter 10. Distal end 14 is provided with a soft distal tip 50, which is not preformed with tubular body 16, but is instead attached to tubular body 16 as a tube post manufacturing step. Distal tip 50 is preferably soft enough and flexible enough, so as to minimize trauma to body vessels as catheter 10 is advanced, and also to facilitate navigation of catheter 10 in tortuous vessels. In one preferred embodiment, distal tip 50 is formed as a 0.5 cm sleeve of 25-40D Pebax (TM), and is bonded to tubular body 16 by heat fusing. Alternately, distal tip 50 may be attached to tubular body 16 by adhesives, or by insert molding, as is known to those of skill in the art. Preferably, distal tip 50 is in alignment with tubular body 16, and does not bend or curve, such that the radial axis of distal tip 50 is substantially the same as that of tubular body 16.

The distal end 14 of catheter 10 is also preferably provided with a radiopaque material 44. Advantageously, radiopaque material 44 serves as a marker to help the clinician position catheter 10 during a medical procedure. Various well-known radiopaque materials may be used in distal end 14, such as platinum, gold, and platinum-iridium blends. The full length, or part of the length of the tubular body, may also be radiopaque by blending radiopaque materials in the polymeric materials used to form the body. Furthermore, radiopacity of the tip can also be achieved by loading (i.e., comparing) the distal tip 50 with a sufficient amount of barium sulfate. Alternatively, bismuth subcarbonate, bismuth trioxide or bismuth oxychloride may be used as a radiopaque filler. Also, radiopacity may be achieved by using radiopaque wire or flat ribbon to make the braid or coil.

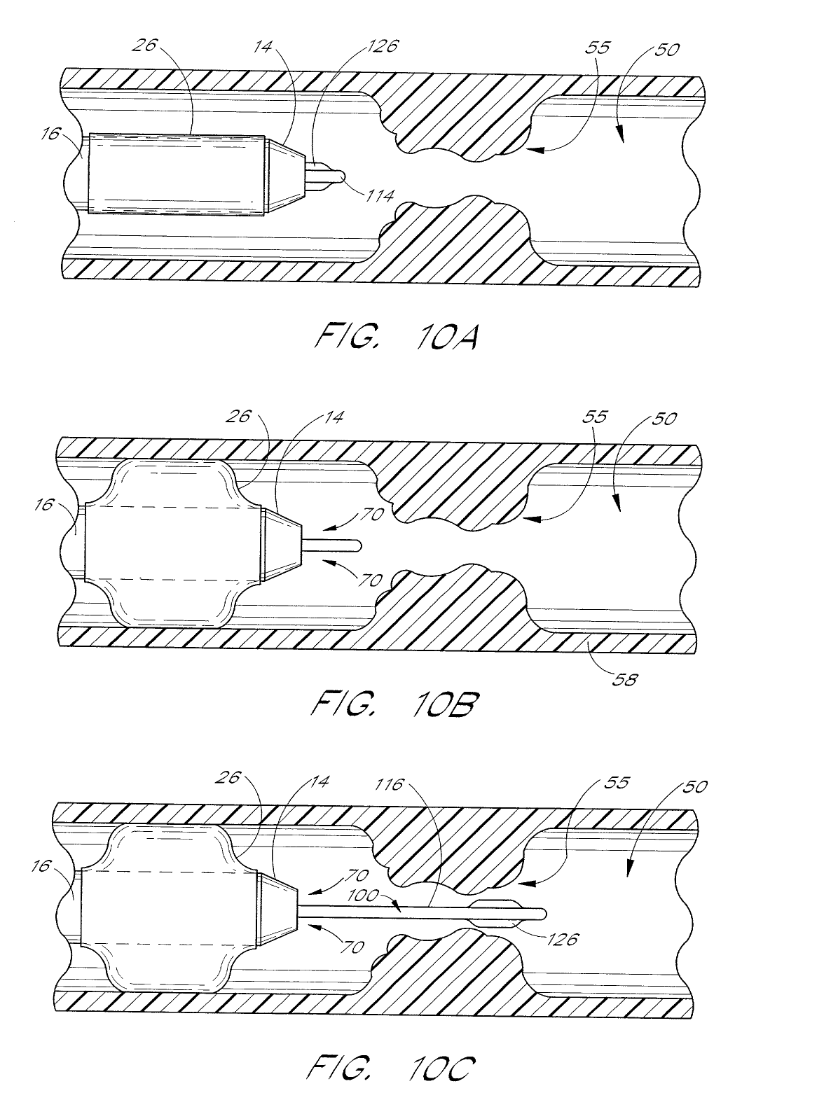

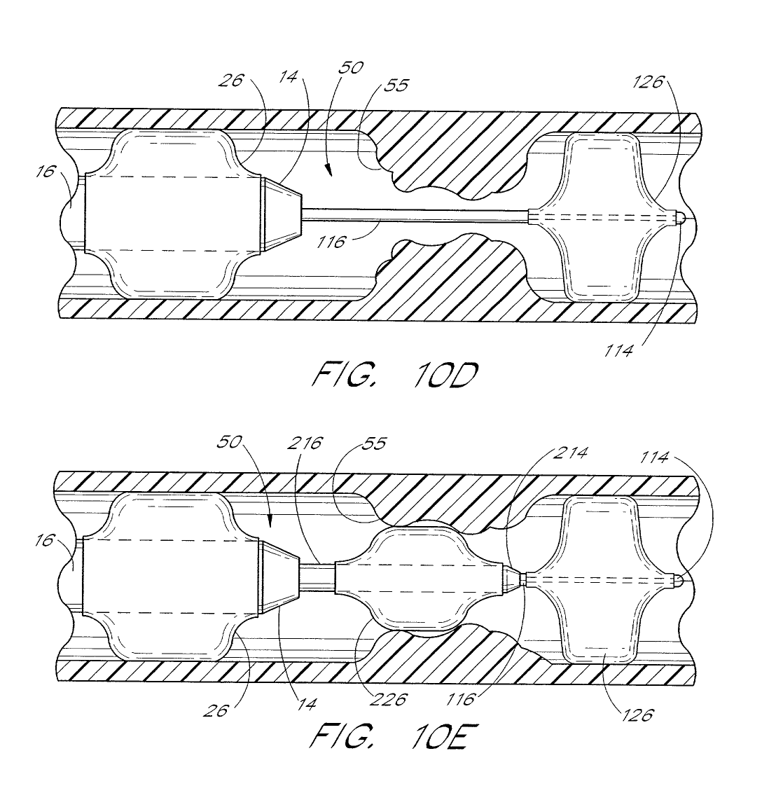

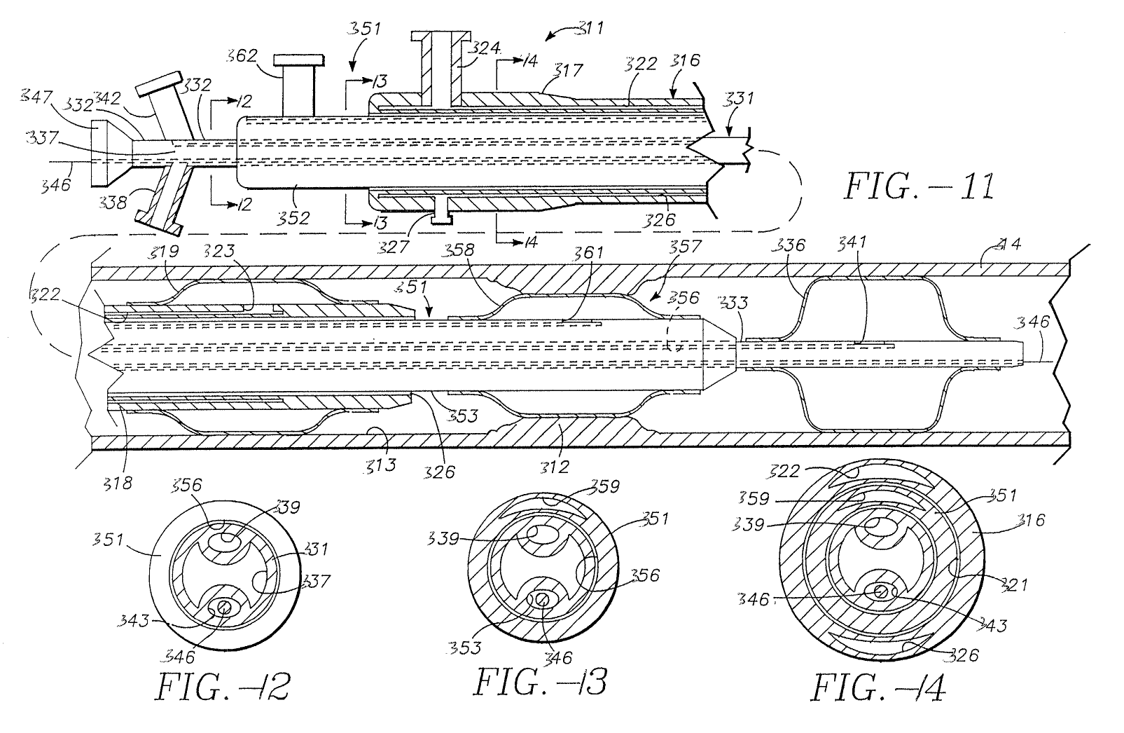

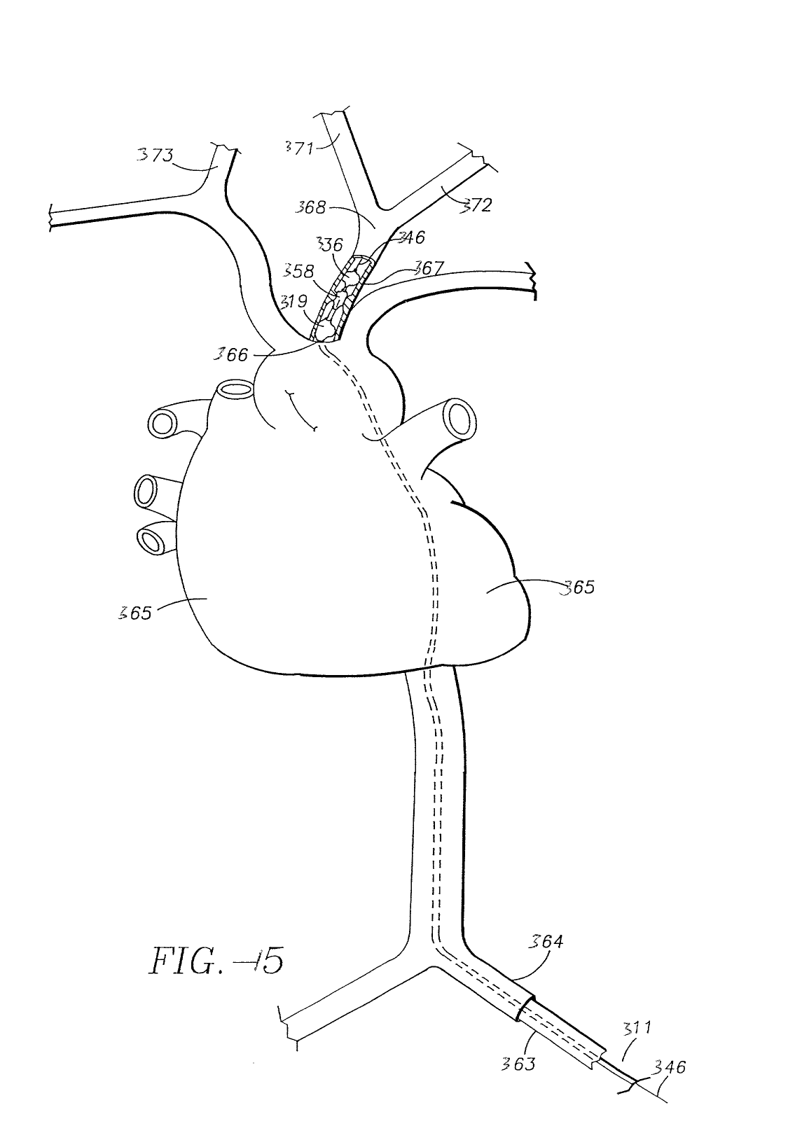

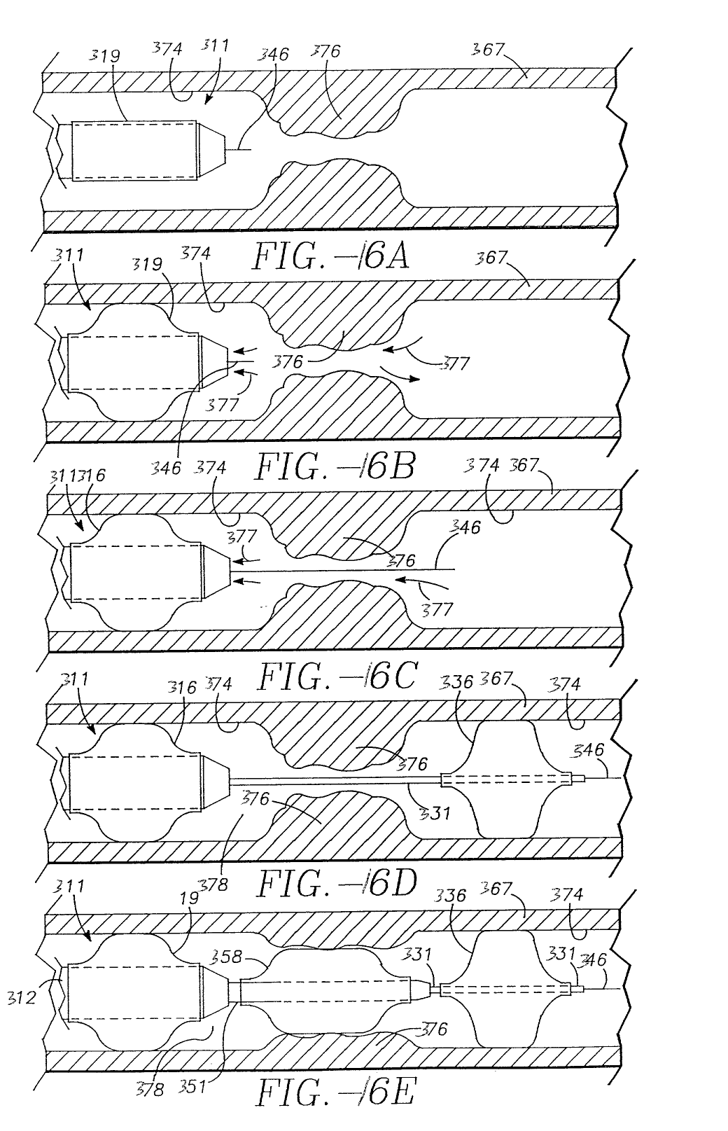

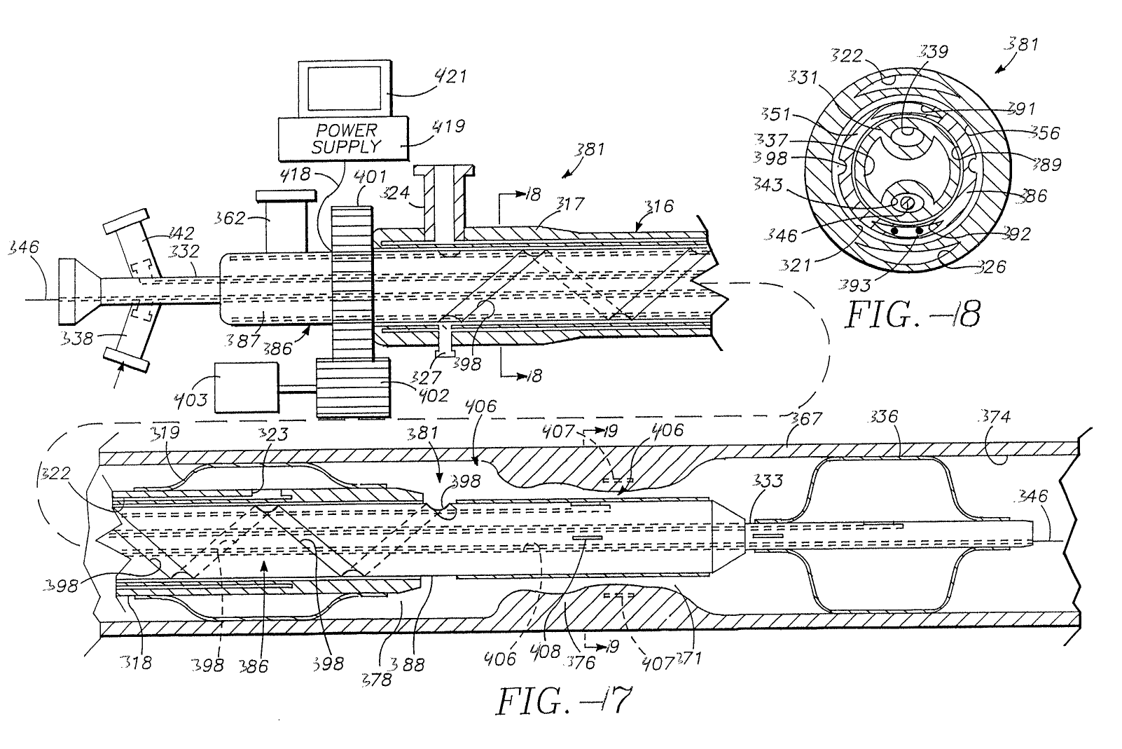

Illustrated in FIGS. 6-9, there is an emboli containment system utilizing catheter 10 of the present invention. Catheter 10 of the present invention is used in the treatment of a stenosis 55 in a lumen 50 in a blood-carrying vessel 58 in which the stenosis 55 at least partially occludes the lumen 50. The emboli containment system depicted in FIG. 6 comprises a catheter 10, as described above, as well as catheters 100 and 200.

Catheter 100 comprises an elongate flexible tubular body 116 having proximal end and distal end 114. An inflatable balloon 126 of the same type as inflatable balloon 26 is coaxially mounted on tubular body 116 on the end 114 of catheter 100. The tubular body 116 has centrally disposed inflation lumen 132 in fluid communication with balloon 126, such that fluid passing through lumen 132 may be used to inflate balloon 126. Alternatively, fluid may be withdrawn from lumen 132 to deflate balloon 126. As shown in FIG. 6, catheter 100 is disposed within main lumen 30 of catheter 10 and is slidably and coaxially mounted therein for variable displacement of balloon 126 with respect to the first balloon 26, as hereinafter described. One preferred embodiment of a catheter 100 is disclosed in our co-pending application, entitled HOLLOW MEDICAL WIRES AND METHODS OF CONSTRUCTING SAME, Ser. No. 08/812,876, filed Mar. 6, 1997, now U.S. Pat. No. 6,068,623, the entirety of which is incorporated by reference.

The emboli containment system also comprises catheter 200 comprising an elongate flexible tubular body 216 having proximal end and distal end 214. Catheter 200 is also provided with a generally centrally disposed lumen 230 extending from the proximal end to the distal end of catheter 200, and through which catheter 100 is coaxially and slidably mounted.

The distal end 214 of catheter 200 is provided with means for performing a medical procedure, such as an apparatus for treating stenotic lesion 55. In the embodiment of the invention shown in FIG. 6, this means comprises a dilatation balloon 226, which is preferably a non-compliant inflatable balloon which is coaxially mounted on the distal end 214 of catheter 200. Balloon 226 may also be attached to tubular body 216 in the same manner as balloons 26 and 126 hereinbefore described. Tubular body 216 is provided with a balloon inflation lumen 232 which is in fluid communication with balloon 226, such that balloon 226 may be inflated by the passage of fluid through lumen 232.

The operation and use of the emboli containment system utilizing the catheter of the present invention for treating occluded vessels may now be briefly described in connection with an occlusion formed by a stenosis in a carotid artery, as illustrated in FIGS. 10A-E.

Catheter 100 is inserted into an incision into a femoral artery of a patient and is advanced through that artery into the aorta of the patient and into the ostium of the carotid artery to be treated. After catheter 100 has been introduced, catheters 10 and 200, with balloons 26 and 226 completely deflated, are introduced over catheter 100 and are advanced into the ostium of the carotid artery and into the lumen or passageway of the vessel as shown in FIGS. 10A-E.

The emboli containment system is advanced until catheter 10 is proximal of a stenosis 55 in the vessel lumen 50 to be treated. Balloon 26 is then inflated by introducing a suitable inflation medium such as a radiopaque liquid into port 18 to cause it to pass through the balloon inflation lumen 32 to inflate balloon 26, as shown in FIG. 10B. Balloon 26 is progressively inflated until it engages the side wall 58 of the vessel to occlude the lumen 50.

Catheter 100 is then advanced through stenosis 55 as shown in FIG. 10C. Catheter 100 with deflated balloon 126 thereon is advanced through stenosis 55 until the balloon 26 is distal of stenosis 55 as shown in FIG. 10D. Balloon 126 is then inflated by passing an inflation medium through lumen 132 to the interior of the balloon 126 to inflate the balloon 126 until it engages the sidewall 58 of the vessel lumen 50. As soon as the balloon 126 has been inflated, a working space is provided between balloons 26 and 126, so that medical procedures can be undertaken to remove or reduce the stenosis 55 in the space between second balloons 26 and 126, without risk of unwanted particles or emboli escaping into the blood stream.

For emboli containment systems featuring balloon dilatation treatment, it is desired to compress the plaque or material forming the stenosis to provide a larger vessel. Thus, catheter 200 is advanced over catheter 100 to cause distal end 214 with balloon 226 thereon to be advanced into the working space. As soon as balloon 226 has been properly positioned within stenosis 55, balloon 226 is inflated with a suitable inflation medium, as for example a radiopaque liquid. Balloon 226 can be inflated to the desired pressure to cause compression of the plaque of the stenosis 55 against the sidewall 58 of lumen 50 by the application of appropriate inflation pressure. As in conventional angioplasty procedures, balloon 226 can be formed of a non-elastic relatively non-compliant material so that appropriate pressures, such as 10-15 atmospheres, can be used within balloon 226 to apply compressive forces to the vessel without danger of rupturing the vessel. It should be appreciated that the non-elastic capabilities can also be achieved by a composite elastic material.

Once the clinician is satisfied that the occlusion forming stenosis 55 has been sufficiently compressed, balloon 226 can be deflated. After the appropriate dilation of stenosis 55 has been accomplished, catheter 200 can be removed from the stenosis. Moreover, in one preferred method, catheter 200 is completely withdrawn from the emboli containment system, and an irrigation catheter is inserted over catheter 100 and through lumen 30, as described in our copending application entitled METHOD FOR EMBOLI CONTAINMENT, Ser. No. 08/812,875, filed Mar. 6, 1997, now U.S. Pat. No. 5,833,644, the entirety of which is incorporated by reference. Fluid introduced into the working space may be removed by supplying a negative pressure or suction to aspiration port 20. This creates a negative pressure within space 30a defined by the interior surface of lumen 30 and outer tubular body 216, to suck or aspirate blood or other fluids in lumen 50 into space 30a and out of aspiration port 20. In this manner, irrigation and aspiration of the working space may take place so that any plaque coming off the occlusion forming the stenosis 55 can be aspirated out of the vessel. Subsequently, balloon 26 and balloon 126 can be deflated to permit normal blood flow through the vessel lumen 50. The entire catheter assembly can then be removed and a suture applied to the incision created to obtain access to the femoral artery.

In general, the catheter apparatus is for treatment of a stenosis in a lumen in a blood carrying vessel. It is comprised of a main catheter and a balloon-on-a-wire device. The main catheter is comprised of a first flexible elongate tubular member having proximal and distal extremities. A first inflatable elastic balloon having an interior is coaxially mounted on the distal extremity of the first flexible elongate tubular member. The first flexible elongate tubular member has a balloon inflation lumen therein in communication with the interior of the first balloon. The first elongate tubular member has a main lumen therein extending from the proximal extremity to the distal extremity and exiting through the distal extremity. An adapter is mounted on the proximal extremity of the first flexible elongate tubular member and has a balloon inflation port in communication with the balloon inflation lumen, a therapeutic catheter port and an aspiration port in communication with the main lumen. The balloon-on-a-wire device is comprised of a guide wire having proximal and distal extremities.

A second inflatable elastic balloon has an interior and is coaxially mounted on the distal extremity of the guide wire. The guide wire has a balloon inflation lumen therein in communication with the interior of the second balloon. The balloon-on-a-wire device is slidably mounted in the therapeutic catheter port and in the main lumen of the first elongate tubular member with the proximal extremity of the guide wire being disposed outside of the main lumen. Removable valve means is carried by the proximal extremity of the guide wire and has the capability of forming a fluid-tight seal with respect to the guide wire while permitting relative axial movement of the guide wire and the first flexible elongate tubular member with respect to each other whereby the first balloon can be moved so that it is proximal of the stenosis and the second balloon so that it is distal of the stenosis. The removable valve means includes an inflation port in communication with the balloon inflation lumen and the guide wire. The apparatus is also comprised of means coupled to the balloon inflation port of the first flexible elongate tubular member for inflating the first balloon and means coupled to the balloon inflation port of the removable valve means for inflating the second balloon to create a working space which brackets the stenosis.

More particularly as shown in FIGS. 11-14, the catheter apparatus 311 of the present invention is for use in the treatment of a stenosis 312 in a lumen 313 in a blood-carrying vessel 314 in which the stenosis 312 has a length and a width or thickness which at least partially occludes the lumen 313. The apparatus consists of a first elongate flexible tubular member 316 formed of a suitable plastic material which is provided with proximal and distal extremities 317 and 318. A first balloon 319 is mounted on the distal extremity 318 and preferably is a compliant balloon formed of a suitable elastic material such as a latex or a very low radiation polyethylene so that it can be inflated to the size of the vessel 314 in which it is to be disposed. Thus, the balloon 319 should be capable of expanding to various diameters depending on the size of the vessel. The first balloon 319 can be formed as a separate balloon separate from the elongate tubular member 316 as shown and adhered thereto by suitable means such as an adhesive (not shown), or it can be formed integral with the tubular member 16 in a manner well known to those skilled in the art.

The tubular member 316 is provided with a large centrally disposed or main lumen 321 extending from the proximal extremity 317 to the distal extremity 318. It is also provided with a balloon inflation lumen 322 which has a distal extremity in communication with the interior of the first balloon 319 through a port 323. The proximal extremity of the balloon inflation lumen 322 is in communication with a balloon inflation fitting 324 mounted on the proximal extremity 317 of the tubular member 316. The fitting 324 can be of a conventional type as for example a Luer-type fitting which is adapted to be connected to a balloon inflation device (not shown) for inflating and deflating the first balloon 319.

The first tubular member 316 is also provided with an aspiration lumen 326 which exits through the distal extremity 318 and the proximal extremity 317 of the tubular member 316. A Luer-type fitting 327 is mounted on the proximal extremity 317 and is in communication with the aspiration lumen 326. The fitting 327 is adapted to be connected to a suitable aspiration or suction source (not shown) of a conventional type such as a syringe or rubber bulb for aspiration purposes as hereinafter described.

The catheter assembly or apparatus 311 also consists of a second elongate flexible tubular member 331 having proximal and distal extremities 332 and 333. A second inflatable balloon 336 of the same type as the first inflatable balloon is coaxially mounted on the distal extremity 333 in a conventional manner. The tubular member 331 is provided with a large generally centrally disposed arterial blood flow lumen 337 which opens through the distal extremity 333 and is in communication with a Luer-type fitting 338 which as hereinafter described is adapted to be connected to a supply of arterial blood from the patient which for example can be taken from another femoral artery of the patient by the use of a blood pump.

The second tubular member 331 is also provided with a balloon inflation lumen 339 which is in communication with the interior of the second inflatable balloon 336 through a port 341. The proximal extremity of the lumen 339 is in communication with the Luer-type fitting 342 mounted on the proximal extremity 332 of the second tubular member 31 and as with the balloon inflation fitting 324 is adapted to be connected to a balloon inflation-deflation device (not shown) of a conventional type. The second tubular member 331 is also provided with a lumen 343 which also can be used as a guide wire and/or for introducing a saline solution extending from the proximal extremity to the distal extremity. The lumen 343 is sized so that it is adapted to receive a conventional guide wire 346 as for example a 0.014″ or 0.018″ guide wire and extends from the proximal extremity to the distal extremity so that the guide wire 346 can extend beyond the distal extremity of the second tubular member 331. A fitting 347 is provided on the proximal extremity 332 in communication with the lumen 343 for introducing the saline solution.

As shown in FIG. 11, the second tubular member 331 is disposed within the central lumen 321 of the first tubular member 316 and is slidably and coaxially mounted therein for displacement of the second balloon 336 with respect to the first balloon 319 as hereinafter described.

The catheter assembly or apparatus 311 also consists of a third elongate flexible tubular member 351 having proximal and distal extremities 352 and 353. It is provided with a centrally disposed lumen 356 extending from the proximal extremity 352 to the distal extremity 353 and through which the second tubular member 331 is coaxially and slidably mounted.

Means 357 is provided on the distal extremity 353 of the third tubular member 351 for performing a medical procedure. In the embodiment of the invention shown in FIG. 11, this means 357 consists of a third balloon 358 which can be non-compliant coaxially mounted on the distal extremity of the third tubular member 351. The third balloon 358 can be attached in the same manner as the first and second balloons 319 and 336 hereinbefore described. The third tubular member 351 is provided with a balloon inflation lumen 359 which has its distal extremity in communication with the interior of the balloon 358 through a port 361. The proximal extremity of the balloon inflation 359 is in communication with a Luer-type fitting 362 provided on the proximal extremity 352 and adapted to be connected to a conventional inflation deflation device (not shown) for inflating and deflating the third balloon 358.

The operation and use of the catheter assembly or apparatus 311 in the method of the present invention for treating occluded vessels may now be briefly described in connection with an occlusion formed by a stenosis in a vessel not having a bifurcation therein as for example in saphenous graft or in one of the right and left carotid arteries, also called internal and external carotid arteries, of a patient in connection with the illustrations shown in FIGS. 15 and 16A-16E. A guiding catheter 363 (FIG. 15) of a conventional type is inserted into an incision into a femoral artery 364 of a patient and is advanced through that artery into the aorta of the heart 365 of the patient and into the ostium 366 of the selected carotid artery or vessel as for example the left carotid 367.

After the guiding catheter has been appropriately positioned, the guide wire 346 is introduced separately into the guiding catheter or along with the catheter assembly 311. The distal extremity of the catheter apparatus or assembly 311 with all of the first, second and third balloons 319, 336 and 358 completely deflated, is introduced into the guiding catheter 363 along with or over the guide wire 346 and is advanced through the guiding catheter 363 into the ostium 366 of the carotid artery or vessel 367 and into the lumen or passageway 368 of the vessel as shown in FIGS. 15 and 16B.

The distal extremity of the catheter assembly 311 is advanced until it is just proximal of a stenosis 369 in the carotid artery 367 to be treated. The balloon 319 is then inflated by introducing a suitable inflation medium such as a radiopaque liquid into the fitting 324 to cause it to pass through the balloon inflation lumen 322 through the port 323 and into the interior of the first balloon 319 to inflate the same as shown in FIG. 16B. The balloon 319 is progressively inflated until it engages the side wall of the vessel 367 to occlude the vessel 367. At the time that this is occurring, a negative pressure or suction is applied to the aspiration fitting 327 to supply a negative pressure through the balloon inflation lumen 322 to suck or aspirate blood in the vessel 367 distal of the first balloon 319 into the aspiration lumen 326 and out the aspiration fitting 327 to thereby reverse the flow of blood through the stenosis as shown by the arrows 371 in FIG. 16B.

While a reverse flow of blood is occurring in the vessel 367, the guide wire 346 is advanced through the stenosis 369 as shown in FIG. 16C. In the event that any pieces or particles of plaque are knocked off of the occlusion formed by the stenosis 369 by movement of the guide wire 346 through the same, such pieces of plaque or emboli will be drawn out with the reverse flow of blood into the aspiration lumen 326 and out of the aspiration fitting 327. During the time that the guide wire 346 is being advanced through the stenosis 369 it may be desirable at the same time to introduce a saline solution through the guide wire lumen 343 of the second elongate flexible tubular member 331 to exit through the distal extremity of the second elongate flexible tubular member 331 into the space immediately proximal of the stenosis 369. This introduced saline solution aids the flow of particulate or other particles dislodged from the stenosis 369 during advancement of the guide wire 346 through the same and carries them back with the mixed saline blood solution through the aspiration lumen 326 in a manner hereinbefore described.

With the guide wire 346 remaining in position, the second elongate flexible tubular member 331 with the second balloon 336 thereon in a deflated condition is advanced over the guide wire 346 through the stenosis 369 until the second balloon 336 is distal of the stenosis 369 as shown in FIG. 16D after which the second balloon 336 is inflated by introducing an inflation medium as for example a radiopaque liquid through the inflation fitting 342 into the lumen 339 through the port 341 to the interior of the second balloon 336 to inflate the second balloon 336 until it engages the sidewall of the vessel 367.

Prior to, during or after inflation of the second balloon 336, the guide wire 346 can be removed. However, it is preferable to remove the guide wire 346 as soon as the second balloon 336 has been advanced so that it is beyond the stenosis 369. At this time, and certainly prior to complete inflation of the second balloon 336, blood is shunted across the stenosis 369 and into the lumen 368 distal of the second balloon 336 by introducing blood through the fitting 338 and into the centrally disposed blood flow lumen 337 in the second tubular member 331 so that it exits out the central lumen 337 distal of the second balloon 336. The blood which is supplied to the fitting 337 can be taken from another femoral artery of the patient and pumped into the fitting 338. In addition, if desired, the blood which is aspirated in the space distal of the first balloon 319 can be appropriately filtered and also supplied to the fitting 338. By shunting blood past the stenosis 369 in this manner it can be seen that blood is being continuously supplied to the carotid artery of the patient during the time that the second balloon 336 is inflated and occludes the lumen 368 in the vessel 367.

As soon as the second balloon 336 has been inflated, it can be seen that there is provided a working space 376 (FIG. 16D) between the first and second balloons 319 and 336 so that medical procedures can be undertaken to remove or reduce the stenosis 369 in the space between the first and second balloons 319 and 336.

Assuming that it is desired to compress the plaque or material forming the stenosis 369 to provide a larger lumen, opening or passageway through the stenosis 369 the third tubular member 51 can be advanced by grasping the proximal extremity 352 to cause the distal extremity with the third balloon 358 thereon to be advanced into the working space 376. As soon as the balloon 358 has been properly positioned within the stenosis 369, the balloon 358 also can be inflated with a suitable inflation medium as for example a radiopaque liquid. The balloon 358 can be inflated to the desired pressure to cause compression of the plaque of the occlusion against the sidewall of the vessel 367 by the application of appropriate pressure. As in conventional angioplasty procedures, the third balloon 358 can be formed of a non-elastic relatively non-compliant material so that high pressures as for example 10-15 atmospheres can be used within the balloon to apply compressive forces to the vessel without danger of rupturing the vessel. It should be appreciated that the non-elastic capabilities can also be achieved by a composite elastic material.

Since the blood flow has been restored to the vessel 367 by the shunt hereinbefore described, the compression of the occlusion forming the stenosis 369 can be carried out for an extended period of time, as for example after a few minutes, if desired to help ensure that a large lumen or passageway is formed through the stenosis 369 as shown in FIG. 16E. If it is believed that the occlusion forming the stenosis 369 has been sufficiently compressed, the third balloon 358 can be deflated. In the event an inelastic balloon is utilized for the third balloon 358, and it is desired to utilize a larger third balloon, this can be accomplished by removing the third tubular member 351 with the deflated balloon 358 thereon and introducing a third tubular member 351 having a larger size balloon thereon over the second tubular member 331 and advancing it into the stenosis 369 and inflating the larger size balloon to create a still larger passage through the stenosis 369.

After the appropriate dilation the stenosis 369 has been accomplished the third balloon can be removed from the stenosis while aspiration of the working space 376 is still ongoing so that any plaque coming off the occlusion forming the stenosis 369 can be aspirated out of the vessel. After the third balloon 358 has been removed from the stenosis, the second balloon 336 and the first balloon 319 can be deflated to permit normal blood flow through the vessel 367 after which the arterial blood flow supply to the fitting 338 can be terminated. The entire catheter assembly 311 can then be removed from the guiding catheter 363 after which the guiding catheter 363 can be removed and a suture applied to the incision created to obtain access to the femoral artery.

In place of the third balloon 358 for causing compression of the occlusion forming the stenosis 367 to create a larger passageway therethrough, an atherectomy device 381 (see FIG. 17) can be utilized for operating in the working space 376 to remove the plaque of the occlusion forming the stenosis. This can be accomplished with a catheter assembly or apparatus 381 which in many respects is similar to the apparatus 311 shown in FIG. 11 and consists of a first tubular member 316 with a first balloon 319 and a second tubular 331 with a second balloon 336 thereon. In place of the third flexible elongate tubular member 351 there is provided a third flexible elongate tubular member 386 which is provided with proximal and distal extremities 387 and 388. The flexible elongate tubular member 386 is slidably and rotatably mounted in the central lumen 321 of the flexible elongate member 316 and is provided with a central or main lumen 389 through which the second flexible elongate tubular member 331 extends. It is also provided with a lumen 391 extending from the proximal extremity to the distal extremity through which a saline solution can be introduced for saline irrigation as hereinafter described. It is also provided with another lumen 392 which is adapted to receive a plurality of electrical conductors 393 for performing electrical functions as hereinafter described. The lumen 392 is connected to a conventional Luer-type fitting 396 serving as a fluid irrigation fitting mounted on the proximal extremity first tubular member 312 and is in communication with an annular recess 397 which is in communication with the lumen 391 provided in the tubular member 386 for supplying a saline irrigation liquid through the flexible elongate tubular member 386 and into the working space 376 provided between the first and second balloons 316 and 336. In order to aid aspiration of the saline irrigation liquid from the working space 376, the outer surface of the flexible elongate tubular member 386 is provided with a helical groove 398 therein which has one end which opens into the working space 376 and which has the other end in communication with the aspiration fitting 327.

Means is provided for rotating the second tubular member 386 and consists of suitable means such as a spur gear 401 mounted on the proximal extremity 387 of the tubular member 386. The spur gear 401 is driven in a suitable manner as for example by another smaller spur gear 402 which is of greater width than spur gear 401 so as to provide a splined gear connection between the gears 401 and 402. This accommodates the desired longitudinal movement for the tubular member 386 so that the distal extremity 388 of the tubular member 386 can be advanced and retracted in the working space 376 as hereinbefore described. An electrical drive motor 403 is provided for driving the gear 402.

Atherectomy means 406 is provided on the distal extremity 388 of the flexible elongate tubular member 386. As shown in FIGS. 17 and 19, the atherectomy means 406 consists of a flexible elongate member 407 formed of a suitable material such as stainless steel or preferably a superelastic Nitinol. The flexible elongate member 407 is wound into a helix as shown in FIG. 19 onto the distal extremity of the tubular member 386. The flexible elongate member 407 can be formed of a ribbon having a thickness of 0.003″ and a width of 0.060″. One end of the flexible elongate member 407 can be secured to the tubular member 386, as for example by inserting the same into a slit 408 and additionally by the use of adhesive (not shown). The flexible elongate member 407 is wrapped into a helix in a direction opposite to the direction of normal rotation of the tubular member 386 and can be provided with a special tip 409 on its free end with the tip having an arcuate surface 411 that is inclined rearwardly to terminate at a cutting edge 412 (see FIG. 19) which is adapted to engage the plaque or the stenosis 369.

When the distal extremity 388 of the flexible elongate tubular member 386 has been introduced into the working space 376, the end or tip 409 of the flexible elongate member 407 of the atherectomy means 406 is free. A saline solution is introduced into the fitting 357. Thereafter the motor 403 can be energized to cause rotation of the tubular member 386 and to thereby cause rotation of the helically wound flexible elongate member 407 to cause its free end or tip 409 to be moved outwardly radially under centrifugal force to bring the cutting edge 412 into engagement with the plaque 369 in the stenosis 369 to cause progressive removal of the plaque forming the stenosis 369 to enlarge the passageway extending through the stenosis. Because of the rounded configuration of the tip 409, the tip 409 will not dig into the vessel wall but will only remove plaque which is engaged by the cutting edge 412. As the plaque is being removed, the saline solution introduced through the fitting 396 into the space 376 picks up the plaque particles or emboli as they are being removed. The saline solution with the plaque or emboli therein is removed through the spiral groove 398 and through the aspiration port 327. The flexible elongate tubular member 386 can be moved back and forth so that the cutting tip 409 engages the length of the stenosis 369 so that substantially all of the stenosis 369 can be removed.

Means is provided to sense when sufficient plaque has been removed from the stenosis 369 and to ensure that cutting edge 412 does not cut into the vessel wall. An ultrasonic sensor 416 (see FIG. 17) is mounted in the distal extremity of the tubular member 386 and is connected by conductors 393 (see FIG. 18) extending through the lumen 392 and connected to a cable 418 which is connected to an ultrasonic power supply 419 and a video monitor 421. By using the Doppler effect, ultrasonic energy can be utilized in connection with the transducer 416 to ascertain the depth of cut being made by the flexible elongate member 407 as it is being rotated.

As soon as a desired amount of plaque has been removed from the stenosis 369 to provide the desired passage through the stenosis, rotation of the tubular member 386 is terminated after which the tubular member 386 can be withdrawn followed by deflation of the second balloon 336 and withdrawing it. Deflation of the first balloon 316 then occurs after which it is withdrawn from the vessel 367. Thereafter, the guiding catheter 363 can be removed and the incision closed as hereinbefore described.

In order to ensure that restenosis will not take place, it may be desirable to place a cylindrical stent 426 in the stenosis 369. Such a stent 426 can be a self-expanding stent formed of a suitable material such as a superelastic Nitinol and movable between unexpanded and expanded conditions. Such a stent 426 can be placed by a suitable catheter apparatus 431 of the type shown in FIG. 20. The stent 426 which is cylindrical in form is pushed over the proximal extremity of the second elongate flexible tubular member 331 into the main or central lumen 321 so that it is retained in the unexpanded position. It is then pushed forwardly toward the distal extremity of the first flexible elongate tubular member 316 by means of a flexible elongate tubular member 436 having proximal and distal extremities 437 and 438 and having a flow passage 439 extending from the proximal extremity 437 to the distal extremity 438. The proximal extremity 437 is provided with a knurled collar 441 which is adapted to be engaged by the hand to facilitate pushing of the flexible elongate tubular member 436 so that its distal extremity is in engagement with the stent 426. Thus, when desired the stent 426 may be discharged or dislodged from the distal extremity of the second tubular member 331 and pushed into the working space 376 created between the first balloon 319 and the second balloon 336.

After the stent 426 has been discharged out of the end of the first flexible elongate tubular member 316, the stent 426 will self expand toward its expanded condition until it is in engagement with the wall of the vessel in the vicinity of the occlusion forming the stenosis 369 to frictionally retain the stent in engagement with the vessel wall. As soon as the stent 426 is in engagement with the vessel wall, the second balloon 336 can be deflated as can the first balloon 319. The first deflated-balloon 336 can then be withdrawn through the interior of the cylindrical stent 426. This can be followed by deflation of the first balloon 319 and the removal of the flexible elongate tubular member 316 with its first balloon 319 and the flexible tubular member 331 with its second balloon 336, along with the flexible elongate member 436 until the entire catheter assembly or apparatus 431 has been removed from the guiding catheter 363. Thereafter the guiding catheter 363 can be removed and the incision sutured as hereinbefore described.

In FIG. 21, there is shown another embodiment of an apparatus 451 incorporating the present invention which is particularly adapted for use treating a stenosis at or near a bifurcation appearing in an arterial vessel. The apparatus 451 is shown being used on a human being 452 showing the principal arteries and pulmonary veins of the human body. Thus there as shown, the abdominal aorta 453 branches into the common iliac 454 which branches into the external iliac 456 and the internal iliac 457. The external iliac branches into the deep femoral artery 458 and into the femoral artery 459. The abdominal aorta 453 extending in the opposite direction passes through the aortic arch 461 of the heart 462. The aortic arch 461 is connected to the common carotid 466 which extends into a bifurcation 467 branching into the external carotid 468 and the internal carotid 469. Similar bifurcations appear in the basilar artery which is an artery which is particularly inaccessible for surgical treatment.

As hereinafter explained, the apparatus 451 shown in FIGS. 21, 22 and 23 consists of a proximal occlusion balloon catheter 476 which can be considered to be a first catheter. The catheter 476 is formed of a flexible elongate tubular member 477 having proximal and distal extremities 478 and 479. The tubular member 477 is formed of a suitable material such as plastic and can have a suitable size ranging from 5 to 14 French and preferably 9 to 10 French. A balloon 481 is provided on the distal extremity 479 and is formed of a suitable elastic material. It is generally cylindrical in form and has its proximal and distal extremities secured to the tubular member 477 by suitable means such as an adhesive (not shown). The tubular member 477 is provided with a plurality of lumens therein. One lumen 482 serves as a balloon inflation lumen and extends from the proximal extremity 478. It can have a suitable size such as 0.024″ and has port 483 in communication with the interior of the balloon 481. A manifold 486 formed of a suitable material such as plastic is mounted on the proximal extremity 478. A tubular member 487 is mounted in the manifold 486 and is in communication with the inflation lumen 482.

The tubular member 477 is also provided with a large lumen 491 having a suitable size as for example 0.045″ which is adapted to slidably receive therein a therapeutic balloon catheter 492 and a perfusion balloon catheter 493. It is also provided with another lumen 496 having a suitable size as for example 0.026″ which is adapted to receive a balloon-on-a-wire catheter 497. It is also provided with an aspiration lumen 501 having a suitable size as for example 0.025″ and an irrigation lumen 502 having a suitable size as for example 0.015″. There is also provided another lumen 503 which can be used for other purposes.

The therapeutic balloon catheter 492 and the perfusion balloon catheter 493 are constructed in a manner similar to the balloon catheters hereinbefore described. Thus the perfusion balloon catheter 493 is provided with a flexible elongate tubular member 506 having proximal and distal extremities 507 and 508. A balloon 509 formed of an elastic material is secured to the distal extremity 508 by suitable means such as an adhesive (not shown) and is adapted to be inflated through a port 510 in communication with a balloon inflation lumen 511. The tubular member 506 is also provided with a blood perfusion lumen 512 which is centrally disposed therein. The proximal extremity 507 of the tubular member 506 is connected to a Y adapter or fitting 513 of which the central arm 514 is in communication with the blood perfusion lumen 512 and is provided with a Luer-type fitting 516. The side arm 517 of the fitting 513 is in communication with the balloon inflation lumen 511 and is provided with a Luer-type fitting 518 adapted to be connected to a source of pressure as hereinafter described.

The therapeutic balloon catheter 492 consists of a tubular member 521 having a proximal and distal extremities 522 and 523. A balloon 524 formed of a non-elastic material is secured to the distal extremity 523 by suitable means such as an adhesive. A port (not shown) is in communication with the interior of the balloon 524 and is in communication with a balloon inflation lumen 526. A Luer-type fitting 527 is mounted on the proximal extremity 522 and is in communication with the balloon inflation lumen 526. Another fitting 528 is mounted on the proximal extremity 522 and is in communication with a large centrally disposed lumen 529 which can receive the perfusion balloon catheter 493 for slidable movement as hereinafter described.

The balloon-on-a-wire catheter 497 is slidably mounted in the lumen 496 and consists of a guide wire 531 of a conventional construction having a suitable diameter as for example 0.018″ and having a proximal and distal extremities 532 and 533. A balloon 534 formed of a non-elastic material is mounted on the distal extremity 533 and is secured thereto by suitable means such as an adhesive (not shown). The proximal extremity of the balloon 534 is secured to the distal extremity of a tubular member 536 formed of a suitable material such as plastic and which is coaxially disposed on the guide wire 531. The tubular member 536 extends the length of the guide wire to the proximal extremity and is connected to a Luer-type wye fitting 537 and is in communication with an annular lumen 538 disposed between the tubular member 536 and the exterior surface of the guide wire 531. The lumen 538 is in communication with the interior of the balloon 534 for inflating and deflating the balloon 534. The balloon-on-a-wire catheter 497 is adapted to be introduced through a fitting 541 carried by a tube 542 mounted in the manifold 486 and in communication with the lumen 496 in the multi-lumen elongate tubular member 477.

A tube 546 is mounted in the manifold 486 and is in communication with the large lumen 491 and is provided with a fitting 547 which is adapted to receive the perfusion balloon catheter 493 and the therapeutic balloon catheter 492 as hereinafter described. Another tube 551 is provided in the manifold 486 and is in communication with the aspiration lumen 501. It is provided with the fitting 552. Another tube fitting 553 is mounted in the manifold 486 and is in communication with the irrigation lumen 502 and is provided with a fitting 554.

The various fittings for the catheter as hereinbefore described are adapted to be connected into a control console 571. The control console 571 consists of a rectangular case 572 which is provided with a front panel 573.

A plurality of balloon inflation deflation devices 576 of a conventional type typically called endoflaters are mounted within the case 572 and have control handles 577 extending through vertically disposed slots 578 provided in the front panel. These endoflaters 576 are labeled as shown in FIG. 21 and are connected by tubing (not shown) through pressure gauges 581 mounted in the front panel 573 and are provided with needle indicators 582 to indicate the pressure being applied by the endoflater to the tubing. The tubing is connected in such a manner so that the endoflater 576 and the associated pressure gauge 581 are connected to a tube 586 which is provided with a mating fitting 587 adapted to mate with a fitting 488 so that it is in communication with the inflation lumen 482 of the proximal occlusion balloon catheter 476. In a similar manner, the tubing 588 is provided with a fitting 589 which mates with a fitting 518 of the balloon inflation lumen 511 of the perfusion balloon catheter 493 for inflating balloon 509. Similarly, tube 591 with its mating fitting 592 is adapted to mate with the fitting 537 for inflating the balloon 534. Similarly, the tube 593 with its fitting 594 mates with the fitting 527 in communication with the balloon inflation lumen 526 for inflating the balloon 524 of the therapeutic catheter 492. Another tube 596 which is provided with its fitting 597 mates with the fitting 552 that is in communication with the aspiration lumen 501. The tube 596 is in communication with the inlet of a blood pump 601 of a suitable type as for example a roller pump well known to those skilled in the art which is mounted within the case 572 and which is connected to a source of electrical power through electrical plug 602 connected into the case 572. The roller pump 601 is provided with an on/off switch 603 mounted on the front panel 573. After it passes through the pump 601, blood is supplied to a blood filter 606 of a conventional type and then is supplied through a tube 611 having a fitting 612 adapted to mate with the fitting 516 of the perfusion balloon catheter which is in communication with the perfusion lumen 512.

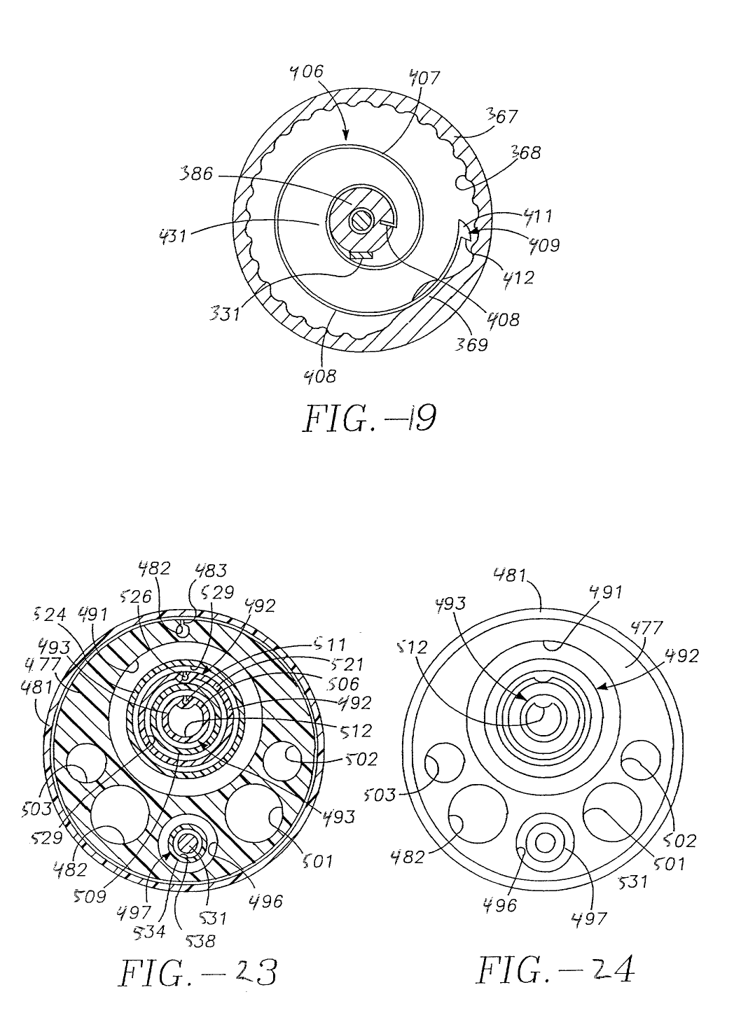

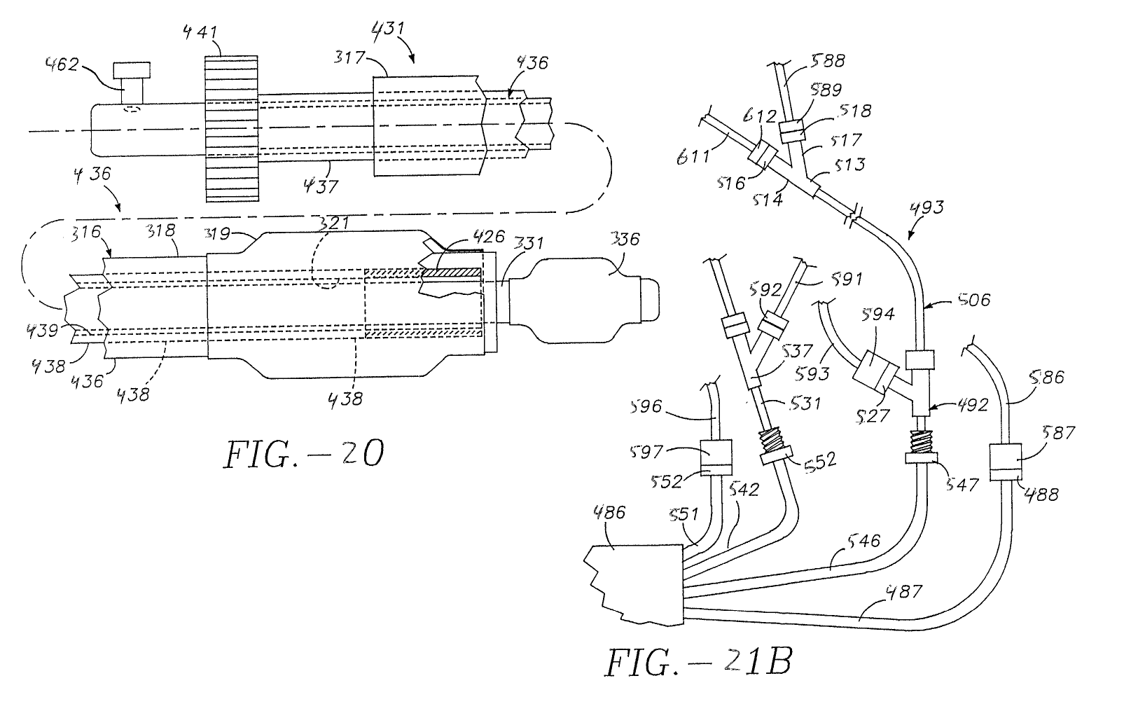

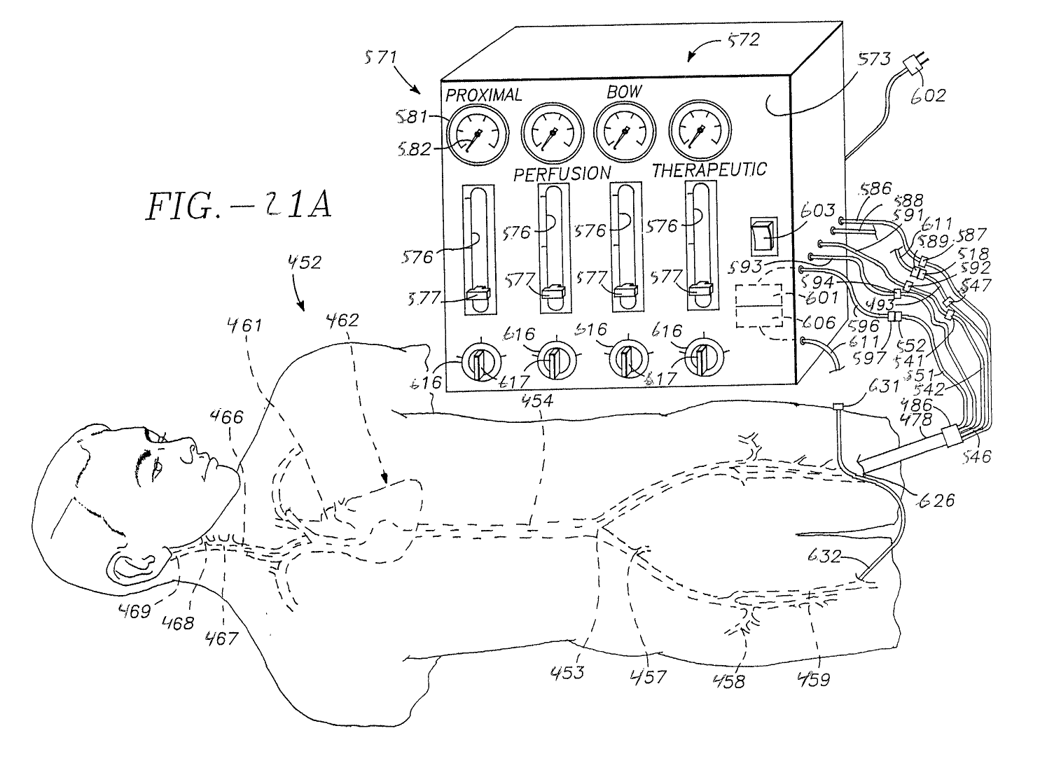

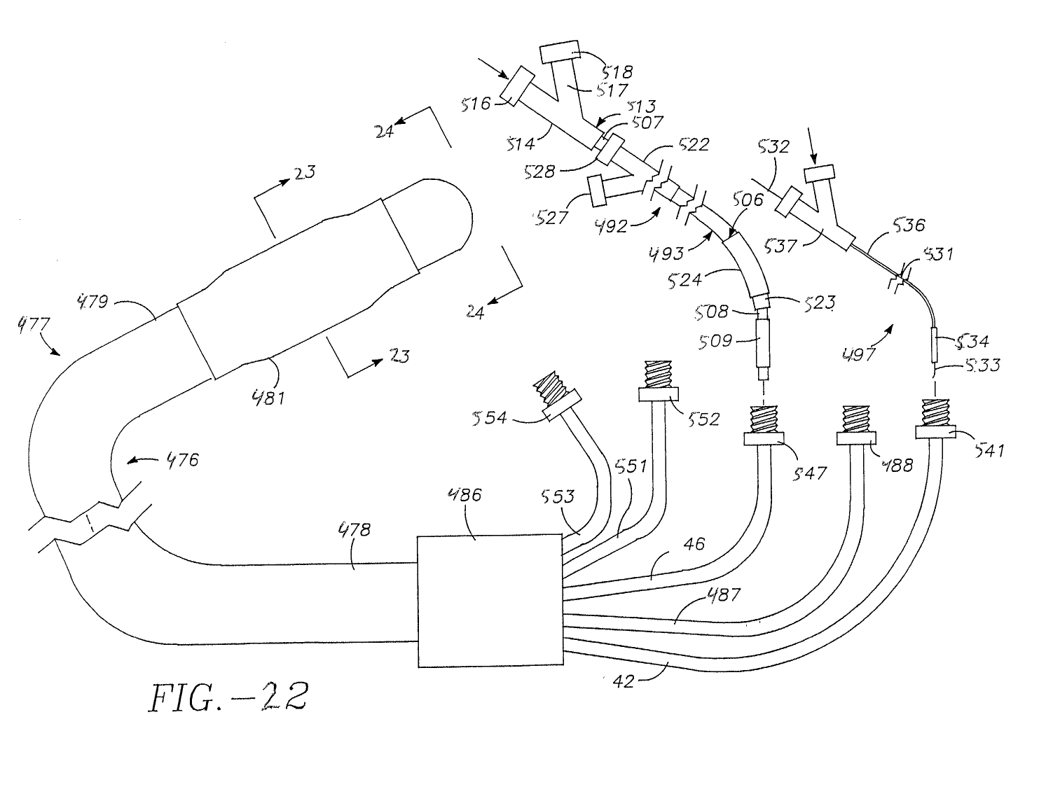

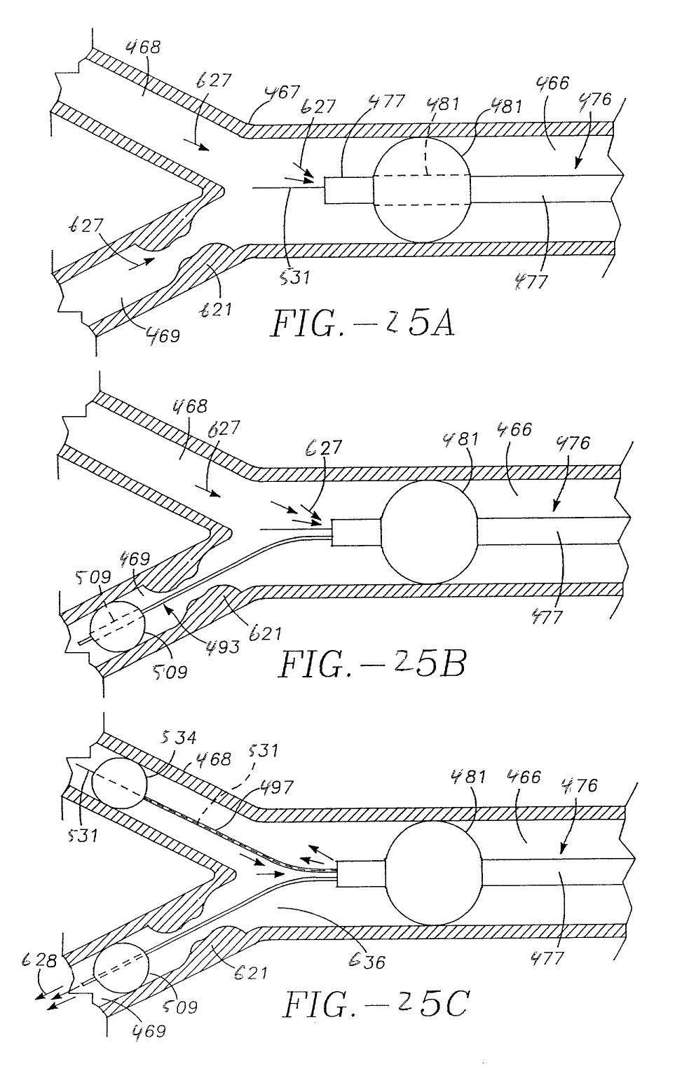

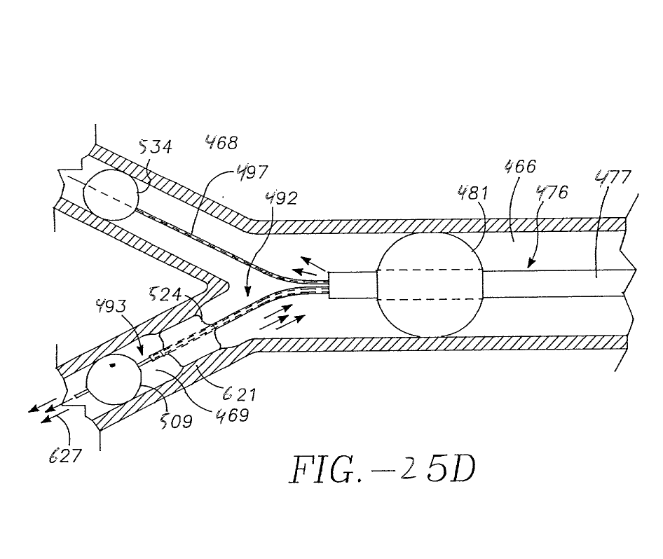

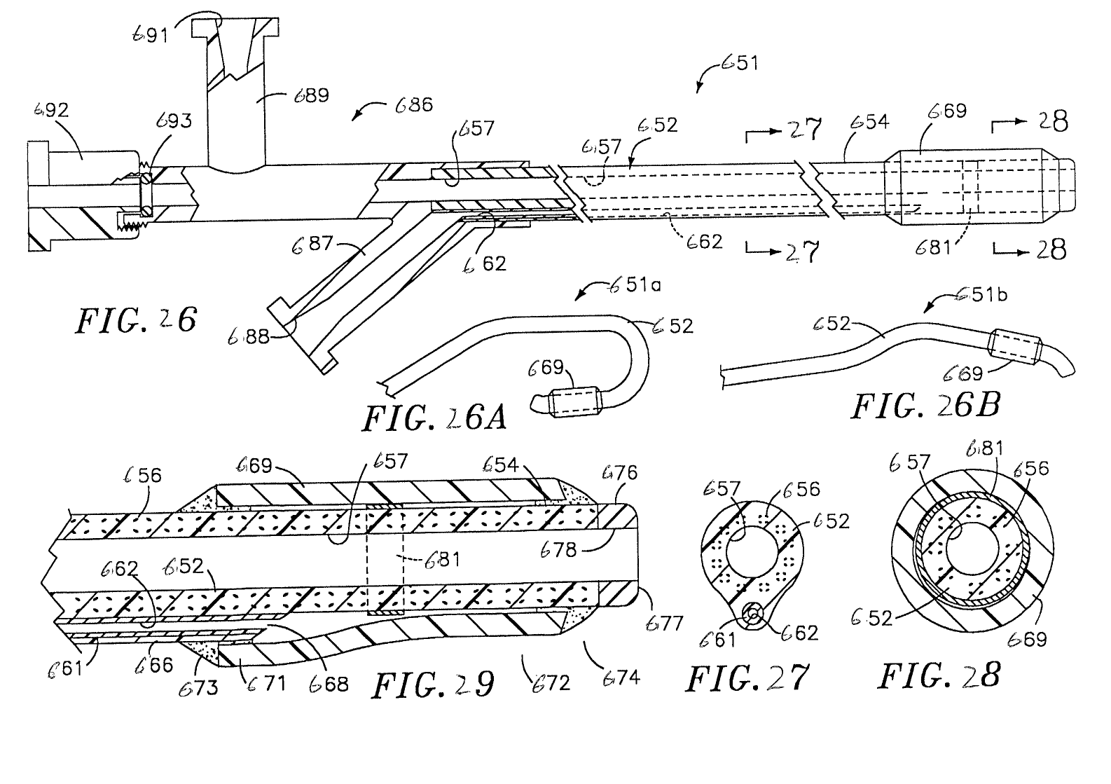

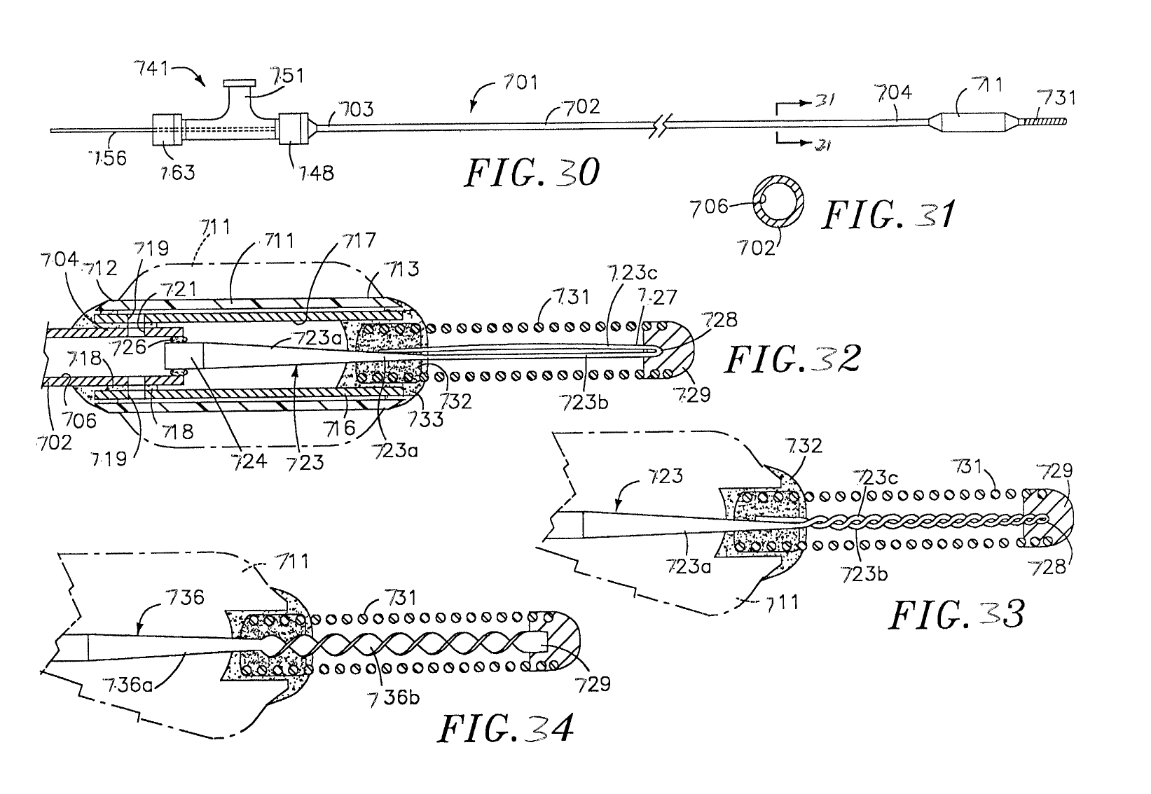

A three-way valve 616 is associated with each of the endoflaters 576 and has a control knob 617 extending through the front panel 573 and is adaptable to be moved between three positions with a center off position and an aspiration position in a counter-clockwise direction and a pressurized position in a clockwise position as viewed in FIG. 24.

Operation and use of the apparatus 451 may now be briefly described as follows. Let it be assumed that it is desired to treat a stenosis occurring in a bifurcation in a carotid artery as depicted by the illustrations shown in FIGS. 25A through 25D. As shown in the illustration in FIG. 25A, let it be assumed that a stenosis is present adjacent the bifurcation 467 and in the external carotid 468 and that it is desired to treat this stenosis in accordance with the apparatus 451 of the present invention in performing the method of the present invention. The proximal occlusion balloon catheter 476 is loaded with the therapeutic balloon catheter 492 slidably mounted over the perfusion balloon catheter 493 and both are slidably mounted in the main lumen 491. The balloon-on-a-wire catheter 497 is slidably mounted in the lumen. While the patient is being prepared for the procedure, all of the lumens in the catheters of the apparatus are flushed with saline to remove all air from the lumens. They are then connected to the control console 571 in the manner hereinbefore described and as shown in FIG. 21. An incision 626 (see FIG. 21A) is made in the femoral artery in the left leg of the patient and a guiding catheter (not shown) similar to the type utilized in angioplasty is introduced through the femoral artery 459. This guiding catheter is advanced until it is near the aorta arch 461. Thereafter, the first or proximal occlusion balloon catheter 476 has its distal extremity 479 introduced into the guiding catheter and advanced in the guiding catheter. It is advanced so that its distal extremity 479 enters the common carotid and is near the bifurcation 467. The balloon 481 is inflated by operating the control handle 577 associated with the proximal occlusion balloon 481 as shown in FIG. 25A to create the desired pressure within and to inflate the elastic balloon 481 so that it occludes the common carotid just proximal of the stenosis 624. As soon as this occurs, the roller pump 601 is turned on by operating the on/off switch 603 to create a negative pressure on the distal side of the balloon 481 to cause blood to flow in a reverse direction as shown by arrows 627 to thereby change the directional flow of blood from the internal and external carotids away from the brain rather than to the brain. The blood travels into the aspiration lumen 501 as indicated by the arrows 627 and into the tube 551 through fittings 552 and 597 and tube 596 to the roller pump 603. The blood after passing through the roller pump 603 passes through a blood filter 606 and then passes into the tube 611 and the fitting 612 and connected to the fitting 589 of the perfusion catheter 493. Alternatively, the fitting 612 can be which is connected to another fitting 631 mounted on a tube 632 introduced into the venous side of the circulatory system of the patient's body, as for example into the vein in the right leg of the patient 452 as shown in FIG. 21. Any debris or emboli in the aspirated blood being pumped will be filtered out by the blood filter 606.

As soon as or during the time this retrograde circulation of blood is established through the roller pump 601, the perfusion balloon catheter 493 extending proximally from the fitting 547 is advanced into the internal carotid 469 past the stenosis 621 at the bifurcation 467. If necessary, a guide wire can be utilized which can be introduced through the perfusion lumen 512 to aide in advancing the perfusion balloon catheter 493 into the internal carotid 469. Any emboli or debris dislodged from the stenosis 621 by crossing the same either by the guide wire or by the distal extremity of the catheter 493 will be picked up by the retrograde flow of blood which is being aspirated through the proximal occlusion balloon catheter 476 to thereby prevent any emboli or debris from entering the brain of the patient. The elastic perfusion balloon 509 is then inflated as shown in FIG. 25B using the appropriate endoflater to inflate the balloon to the desired pressure while watching the associated pressure gauge. As soon as occlusion occurs, perfusion of blood can be started as hereinafter described.

Prior to or after the balloon 509 of perfusion catheter 493 has been inflated, the balloon-on-a-wire catheter 497 extending proximally of the fitting 541 is advanced into the external carotid 469 as shown in FIG. 25C. The balloon 534 is then expanded by use of the appropriate endoflater to supply an inflating medium through the fitting 537 to occlude the external carotid 469. As soon as occlusion has been accomplished in both the external and internal carotids, retrograde flow of blood is terminated by shutting off the roller pump 601. It should be appreciated that if desired, automatic controls can be provided whereby when a certain pressure is reached in each of the balloons 509 and 534 the roller pump would automatically be shut off to stop retrograde flow. By this procedure, it can be seen that the lesion of stenosis 621 has been bracketed by the balloons 481, 509 and 534. Prior to that occurring, retrograde flow of blood is established to prevent any emboli or debris from moving towards the brain.

As soon as retrograde flow of blood has been terminated, perfusion of blood is started. This can be accomplished by connecting a cannula (not shown) to the fitting 516 of the perfusion catheter 506 and to obtain a supply of blood from the femoral artery in the other leg of the patient. Alternatively, an outside blood supply can be used. Thus fresh blood will be supplied from the femoral artery of the patient directly into the perfusion balloon so that it is discharged distally of the perfusion balloon 509 as shown by the arrows 628 to continue to supply blood to the carotid artery. It has been found that it is unnecessary to a supply perfusion of blood to the external carotid artery because there is sufficient auxiliary circulation in that carotid artery during the time the procedure is taking place.

In the event there is inadequate pressure on the arterial blood being perfused to overcome the resistance in the lumen 469, the roller pump 601 can be utilized by merely operating the same in a reverse direction and connecting it between the cannula and the perfusion catheter.

After the lesion or stenosis 621 has been bracketed as hereinbefore described and a working space 636 formed adjacent the stenosis or lesion 621, a therapeutic procedure can be employed. By way of example this can consist of advancing the therapeutic balloon catheter 492 over and axially of the perfusion catheter 493 to bring its balloon 524 into registration with the stenosis 621 as shown in FIG. 25D. Thereafter, the balloon 524 can be inflated by use of the appropriate endoflater as hereinbefore described to cause the inelastic balloon to be pressurized to a pressure of 10 to 15 atmospheres to compress the stenosis 621. Prior to or during this procedure it may be desirable to introduce a saline or heparin solution or a radiopaque contrast liquid into the working space 636. This can be accomplished by introducing this liquid through the injection lumen 502. If desired, this can be accomplished prior to terminating the aspiration procedure hereinbefore described. Also it should be appreciated that if desired a small endoscope can be inserted through one of the lumens to view the area within the working space. Alternatively, if desired an ultrasonic probe can be utilized to view the area in which the lesion is disposed.

As hereinbefore described with a previous embodiment, in place of the therapeutic balloon catheter, other types of catheters can be utilized as for example one incorporating an atherectomy device of the type hereinbefore described to facilitate removal of the stenosis. It is readily apparent that during these procedures if it is necessary to supply a saline solution or a heparinized solution into the working space that the working space can also be continued to be aspirated to remove any debris or emboli which occur during the procedure.

Let it be assumed that the desired therapeutic actions have been undertaken and that the stenosis 621 has been reduced and substantially eliminated so that there is adequate flow through the internal carotid. If it can be seen that there also is a stenosis in the external carotid, the balloon-on-a-wire catheter 497 and the perfusion catheter 493 can be withdrawn and moved so that they enter the opposite carotid to permit therapeutic treatment of a stenosis occurring in the other carotid.

When all the desired therapeutic procedures have been accomplished, the supply of saline or contrast solution can be terminated and the therapeutic balloon 524 deflated. The balloon 534 of the balloon-on-a-wire catheter can be deflated as well as the perfusion balloon 509. Perfusion of blood through the perfusion catheter can be terminated. The perfusion balloon catheter 493 and the balloon-on-a-wire catheter 497 can be retracted into the main multi-lumen tubular member 477 of the proximal occlusion balloon catheter after which the perfusion balloon catheter can be withdrawn carrying with it the other catheters disposed therein. Thereafter, the guiding catheter can be removed and a suture applied to the incision made to gain access to the femoral artery.

It is readily apparent that similar procedures can be carried out with respect to other vessels in the body, such as saphenous vein grafts in the heart, and particularly with respect to vessels in the brain where it is difficult if not impossible to employ surgical procedures as for example with respect to the basilar arteries in which bifurcations appear.

As also herein before explained, the catheter apparatus of the present invention can be utilized for deploying stents. Where that is desirable the apparatus of the present invention, perfusion can be accomplished during employment of the stent.

From the foregoing it can be seen that an apparatus and method has been provided for treating occluded vessels and particularly for treating carotid arteries. The apparatus and method of the present invention is particularly advantageous for the carotid arteries because it permits access to portions of the carotid arteries which are not accessible by surgery.

The catheter apparatus assembly and method of the present invention are also particularly useful for treating other occluded vessels but particularly the carotid arteries because it makes possible the removal of plaque without endangering the patient. An operating or working space is provided while shunting blood around the working space so that there is continued blood flow in the vessel to support the functions which are normally supported by the vessel. As also pointed out above, the apparatus and method of the present invention are particularly useful in connection with vessels having bifurcations therein and in which the stenosis occurs at or near the bifurcation. From the foregoing it can be seen with the apparatus and method of the present invention, retrograde flow of blood is accomplished during deployment of the device to prevent undesired travel of emboli. Occlusion of the vessels is provided to obtain a working space by bracketing the working space with balloons while at the same time maintaining perfusion of blood making it possible to utilize a substantial period of time for undertaking therapeutic procedures with respect to the bracketed stenosis.

In connection with the present apparatus and method for treating occluded vessels, it has been found that it is possible to utilize the apparatus and method without perfusion and other procedures involving the carotid arteries and saphenous vein grafts for periods of time extending over five minutes and greater which has made it possible to simplify the apparatus and the method utilized in conjunction therewith.

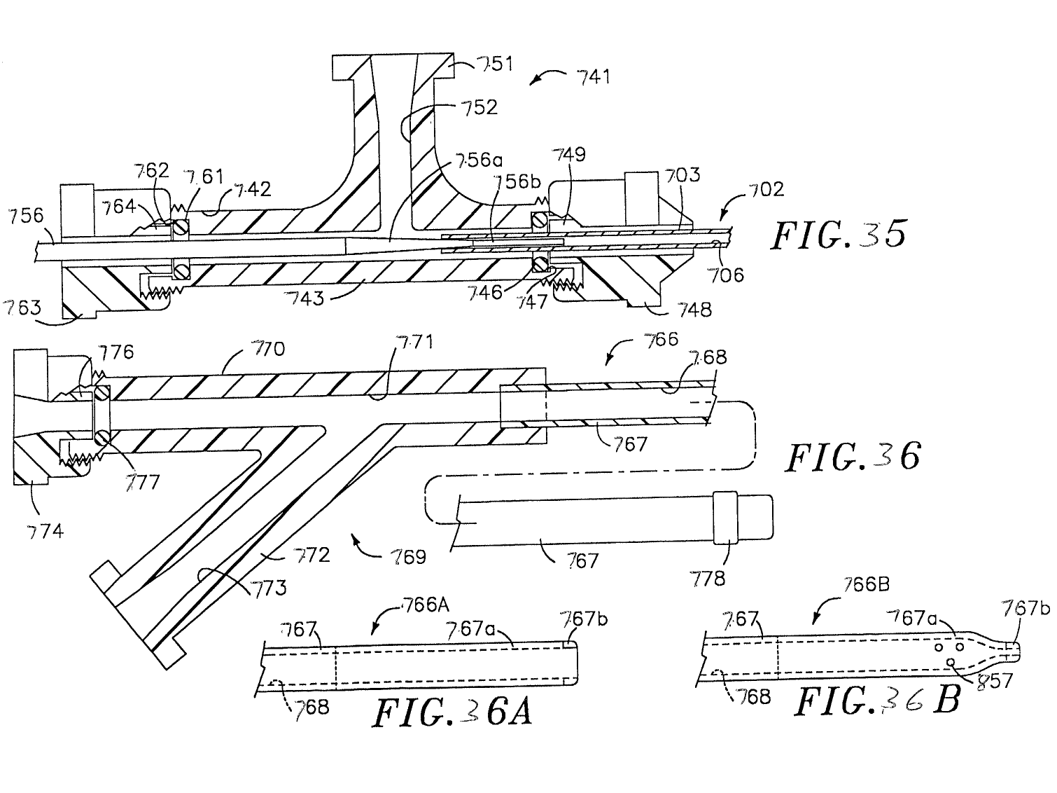

With respect to an apparatus or assembly which does not require the use of perfusion, a main catheter 651 utilized as a part of the apparatus is shown in FIGS. 26, 27, 28 and 29 consists of a flexible elongate tubular member 652 formed of a suitable material such as plastic of the type hereinbefore described and which has proximal and distal extremities 653 and 654. The tubular flexible elongate tubular member 652 can be of various sizes as for example for a saphenous vein graft catheter it can be 8 to 9.5 French in balloon profile with a length ranging from 80 cm to 120 cm. The flexible elongate tubular member 652 can be formed of a suitable material such as PEBAX, Nylon, Hytrel, polyurethane or polyethylene. A flexible braid 656 (see FIGS. 27, 28 and 29) formed of a suitable material such as stainless steel is embedded within the wall of the flexible elongate tubular member 652 as shown and extends from the proximal extremity 653 to the distal extremity 654. The braid 656 can be formed of a suitable stainless steel such as a wire or ribbon having a thickness of 0.001″. The braid 656 provides additional torquability and also inhibits the kinking of the flexible elongate tubular member 652 when it must extend over a tight radius. The flexible elongate tubular member 652 is provided with a large central lumen 657 having a suitable diameter such as 0.065 or greater” extending from the proximal extremity to the distal extremity.

If it is desired to provide a flexible elongate member 652 which has a greater flexibility at the distal extremity, a different material can be used in the distal extremity 654. For example, the distalmost 5-15 centimeters can be formed of a material such as PEBAX having a Shore D hardness of 35-50 with the remainder of the flexible elongate member 652 having a Shore D hardness of 65-75.

A supplemental flexible elongate tubular member 661 is provided which has incorporated therein a balloon inflation lumen 662. The supplemental flexible elongate tubular member 661 can be of a suitable size as for example an I.D. of 0.014″ and an O.D. of 0.018″ and formed of a suitable material such as a polyimide. The supplemental flexible elongate tubular member has a length which is almost as long as the flexible elongate tubular member 652 and overlies the outside wall of the flexible elongate tubular member 652 and extends from the proximal extremity to near the distal extremity as shown in FIGS. 26 and 29. A tube 663 of a suitable material such as Pebax extends over the length of the polyimide tubing 661 and is secured to the flexible elongate tubular member 652 by a shrink tube 666 extending from the proximal extremity 653 to the distal extremity 654, after which the shrink tube 663 is subjected to heat. The shrink tube 666 is then subjected to a hot melt process of a temperature around 350° F. for a period of time until the Pebax tube 663 melts, after which the shrink tubing 666 can be stripped off so that there remains a relatively uniform mass formed of Pebax that surrounds the braid 657 and the polyimide tube 661 which forms the supplemental flexible elongate tubular member 661. The polyimide tube which forms the supplemental flexible elongate tubular member 661 thus provides an inflation lumen 667 extending from the proximal extremity and to the distal extremity and opens through an opening 668 into the interior of an occlusion balloon 669 which is bonded to and coaxially mounted on the distal extremity of the flexible elongate member 652 in the manner shown in FIG. 29. The polyimide tubing is provided to give the balloon inflation lumen shaft 361 greater strength than that which is provided by the Pebax itself.

As can be seen from FIG. 29, the supplemental flexible elongate tubular member 661 is terminated short of the distalmost extremity of the flexible elongate tubular member 652 by approximately 1 cm. The occlusion balloon 669 is formed of various compliant or non-compliant materials. Suitable compliant materials include elastomers such as C-Flex latex, silicones and polyurethanes. Suitable non-compliant materials would be polyethylene, PET and Nylon. A composite material can also be used such as a combination of PET and an elastomer. The occlusion balloon 669 should have a strength so that it can readily accommodate any pressure of one atmosphere and as high as four atmospheres, or approximately 60 psi. The occlusion balloon 669 is cylindrical and is provided with proximal and distal extremities 671 and 672 which are secured by a suitable medical grade adhesive. Alternatively, fuse bonding may be used. Thus a seal 673 formed of this adhesive bonds the proximal extremity 671 of the occlusion balloon 669 over the outer surface of the distal extremity of the flexible elongate tubular member 652 and the supplemental flexible elongate tubular member 661. Similarly, a seal 674 bonds the distal extremity 672 to the distal extremity of the flexible elongate tubular member 652 to provide an air-tight space within the balloon accessible through the opening 668. A soft cylindrical tip 676 formed of suitable material such as Pebax is bonded to the distal extremity of the flexible elongate tubular member 652 and is provided with a rounded surface 677 which extends forwardly and has a passage 678 therein in communication with the lumen 657 and the flexible elongate tubular member 652. A cylindrical radiopaque marker 681 formed of a suitable material such as platinum, platinum-iridium or gold is mounted on the distal extremity of the flexible elongate tubular member 652 in a position so it is substantially equidistant of the ends of the occlusion balloon 669.

A main adapter or fitting 686 formed of a suitable material such as plastic is mounted on the proximal extremity 653 of the flexible elongate tubular member 652. It is provided with a first Luer fitting 687 which provides a balloon inflation port 688 in communication with the balloon inflation lumen 662. It is also provided with another Luer fitting 689 which is provided with an aspiration port 691 in communication with the main central lumen 657. The main adapter 686 is also provided with a Tuohy-Borst fitting 692 which is in communication with the central lumen 657. The Tuohy-Borst fitting 692 is adapted to receive therapeutic devices, as for example a balloon-on-a-wire device as hereinafter described and is adapted to form a liquid-tight seal therewith by an o-ring 693.

A balloon-on-a-wire device 701 incorporating the present invention is shown in FIGS. 30 and 31. The device 701 consists of a guide wire 702 formed of a suitable material such as stainless steel and having a suitable diameter as for example ranging from 0.010″ to 0.032″ but preferably a diameter ranging from 0.014″ to 0.018″. It is preferable that the guide wire 702 be formed of a nickel titanium alloy typically called Nitinol which has the advantage that it is more flexible and has greater kink resistance characteristics than another suitable material such as stainless steel.

It has a suitable length as for example 150 cm. The guide wire 702 is provided with proximal and distal extremities 703 and 704 and is provided with a central lumen 706 extending from the proximal extremity to the distal extremity. The lumen can be of a suitable size as for example 0.010″ I.D. for an 0.014″ O.D. guide wire.

An occlusion balloon 711 is coaxially mounted on the distal extremity 704 of the guide wire 702. The occlusion balloon 711 is preferably formed of the same material as the occlusion balloon 669 on the main catheter 651. The occlusion balloon 711 has proximal and distal extremities 712 and 713. A tube 716 formed of a suitable material such as a polyimide is disposed within the occlusion balloon 711 and has a bore 717 extending therethrough which is sized so that it is slightly larger than the outside diameter of the guide wire 702 so that its proximal extremity can be slipped over the distal extremity 704 of the guide wire 702 and then bonded thereto by suitable means such as an adhesive 718. A plurality of circumferentially spaced apart radially extending inflation holes 719 are provided in the proximal extremity of the tube 716 and are in alignment with similarly spaced holes 721 provided in the distal extremity 704 of the guide wire 702 so that they are in communication with the central lumen 706 of the guide wire 702. The inflation holes 719 as shown are in communication with the interior of the occlusion balloon 711 so that fluid passing from the passage 706 can be utilized for inflating the occlusion balloon 711.

A solid core wire 723 formed of a suitable material such as stainless steel is provided with a proximal tapered extremity 724. The core wire 723 is sized so it is adapted to fit within the lumen 706 of the guide wire 702 and is secured therein by suitable means such as an adhesive 726 or alternatively a weld. The core wire 723 has a tapered portion 723a which commences at the proximal extremity 724 and which is tapered so that the cross-sectional diameter progressively decreases to the distal extremity of the occlusion balloon 711. The core wire 723 is also provided with additional portions 723b and 723c which can be of substantially constant diameter as for example 0.003″. The portion 723 is folded over with respect to the portion 723b so that the portions 723b and 723c lie in a plane to facilitate shaping of the distal extremity of the guide wire 702 during use of the same. The core wire 723 is provided with a distal extremity 727 in which a bend 728 is formed between the two portions 723b and 723c. The bend 728 is secured within a hemispherical solder bump or protrusion 729 which is carried by the distal extremity of a coil 731 formed of a suitable radiopaque material such as platinum or a platinum alloy. The platinum coil 731 can have a suitable outside diameter as for example 0.014″ corresponding to the diameter of the guide wire 702 and can have a suitable length ranging from 1 to 3 cm. The proximal extremity of the coil 731 is secured to the distal extremity of the polyimide tube 716 by suitable means such as an adhesive 732 which can be the same adhesive or a different adhesive 733 utilized for securing the distal extremity 713 of the balloon to the polyimide tube 716 to form a fluid-tight seal between the distal extremity of the occlusion balloon 711 and the distal extremity of the polyimide 716. From this construction it can be seen that the portions 723b and 723c of the core wire 723 in addition to serving as a shaping ribbon are also utilized as a safety ribbon to ensure that the tip 728 and the spring 731 cannot be separated from the guide wire 702. The proximal extremity 712 of the balloon 711 is also secured to the proximal extremity of the polyimide tube 716 and also to the distal extremity 704 of the guide wire 702 to form a fluid-tight seal with respect to the occlusion balloon 711 so that the occlusion balloon 711 can be inflated and deflated through the inflation holes 719 and 721.

Alternative constructions for the distal extremity of the core wire 723 are shown in FIGS. 33 and 34. In FIG. 23 it can be seen that the portions 723b and 723c have been twisted to in effect provide a twisted pair serving as a safety ribbon and as a shaping ribbon. In the embodiment shown in FIG. 34, the core wire 736 is provided with a tapered portion 736a which is the same as the tapered portions of 723a hereinbefore described. However, the core wire 736 has been provided with a distal portion 736b which has been flattened to a suitable thickness as for example a width of 0.006″ and a thickness of 0.003″ and then twisted to form a helix as shown in which the distal extremity is embedded within the solder 729. Such a helix 736 can serve as a safety ribbon and also can be shaped to some extent.

A removable inflation fitting 741 or valve attachment 741 is mounted on the proximal extremity of the guide wire 702 and forms a part of the balloon-on-a-wire device 701. The fitting or attachment 741 is formed of a suitable material such as a polycarbonate and is provided with a central bore 742. The attachment or fitting is slid externally over the proximal extremity 703 of the guide wire 702. Means is provided for forming a fluid-tight seal between the proximal extremity 703 of the guide wire 702 and a body 743 of the fitting 741 and consists of an o-ring 746 (see FIG. 35) seated in a well 747. A thumb screw 748 is threadedly mounted on the body 743 and is provided with an inwardly extending circular protrusion 749 that is adapted to engage the o-ring 746 and to compress the same to form a fluid-tight seal when the protrusion 749 is moved inwardly toward the o-ring 746 as the thumb screw 748 is rotated in a clockwise direction. The o-ring 746 decompresses or springs back when released upon rotation of the thumb screw 748 in a counterclockwise direction so that the fitting 741 can be removed from the distal extremity 703 of the guide wire 702. The body 742 also includes a Luer fitting 751 which provides an inflation port 752 that is in communication with the bore 742 in the body 743 and which is also in communication with the open proximal extremity of the guide wire 702 and the lumen 706 therein.

Means is provided for plugging the bore 706 when the removable attachment or fitting 741 is removed and consists of a plug mandrel 756 formed of a suitable material such as 0.014″ stainless steel solid rod. It is necessary that this rod have a diameter which is greater than the diameter of the lumen 706 and the guide wire 702. The plug mandrel 756 is provided with a progressive portion 756a that tapers down from as, for example from 0.014″ to a suitable diameter as for example 0.008″ to a cylindrical portion 756b.

Means is provided for forming a fluid-tight seal between the plug mandrel 756 which forms a plug mandrel and the body 743 of the attachment or fitting 741 and consists of an o-ring 766 providing suitable sealing means seated within a well 762 provided in the body 743. A thumb screw 763 threadedly engages the body 743 and is provided with a cylindrical protrusion 764 which engages the o-ring and compresses it to form a fluid-tight seal with respect to the plug mandrel 756 by rotation in a clockwise direction of the thumb screw 763. The plug mandrel 756 can be released by a counterclockwise rotation of the thumb screw 763 permitting decompression of the o-ring 761.

An irrigation catheter 766 incorporating the present invention is shown in FIG. 36 and consists of a flexible elongate tube 767 formed of a suitable material such as polyethylene, PEBAX, Hytrel or Teflon having a suitable size as for example an outside diameter of 0.066″ and an inside diameter of 0.058″ and having a length of approximately 150 cm. A lumen 768 is provided therein and extends from the proximal extremity to the distal extremity and is in communication with an adapter 769 provided on the proximal extremity of the tube 767. The adapter 769 is provided with a body 770 formed of a suitable material such as plastic and is provided with a bore 771 extending therethrough. The adapter 769 is provided with a side arm 772 which carries a conventional Luer-type connection and provides an irrigation port 773 in communication with the bore 771. A thumb screw 774 threadedly mounted on the body 770 carries a cylindrical protrusion 776 adapted to compress an o-ring 777 carried by the body 770 into engagement with a therapeutic catheter of the type hereinafter described. A radiopaque tip marker 778 of a suitable type, as for example one formed as a platinum-iridium band 778 is provided on the distal extremity of the flexible elongate element 767 to facilitate positioning of the irrigation catheter as hereinafter described.

Operation of the apparatus shown in FIGS. 26 through 36 in performing the method of the present invention for treating occluded vessels may now be briefly described as follows utilizing the cartoons which are shown in FIGS. 37-43. Let it be assumed that it is desired to treat a vessel 781 in the human body as for example a saphenous vein graft having at least a partial occlusion or stenosis 782 which is formed by plaque in the vessel. The main catheter 651 is introduced into the body through a conventional procedure such as for example by making an incision into the femoral artery in a leg of the patient.

Thereafter the main catheter 651 can be introduced into the femoral artery by use of a large conventional guiding catheter because the main catheter 651 is of a relatively large size, as for example 8 to 9.5 French. In order to eliminate the need for such a large guiding catheter, a smaller conventional guiding catheter 786 of the type shown in FIG. 37 can be utilized which can be introduced through the main catheter 651. Utilizing such a catheter, the main catheter 651 can be inserted independently through a conventional sheath (not shown) in the femoral artery and thereafter the guiding catheter 786 is introduced through the main catheter 651 so that its distal extremity 789 is in the vessel. Alternatively, the guiding catheter 786 can be deployed into the main catheter 651 and the guiding catheter 786 introduced at the same time into the femoral artery.

The guiding catheter 786 is conventional and thus will not be described in detail. It consists of a flexible elongate tubular member 787 (see FIG. 37) formed of a suitable material such as plastic having proximal and distal extremities 788 and 789. The distal extremity 789 is provided with a preformed bend as shown. An adapter 792 is mounted on the proximal extremity 788 and consists of a body 793 in the form a wye in which the central leg 794 is provided with a flow passage (not shown) therein in communication with the central lumen (not shown) extending from the proximal extremity 788 to the distal extremity 789 of the flexible elongate tubular member 787. The body 793 is provided with a side leg 796 which also is in communication with a lumen (not shown) extending from the proximal extremity 788 to the distal extremity 789. A knob 797 carrying an o-ring (not shown) secures the adapter 792 to the proximal extremity 788 with a fluid-tight seal. Another knob 798 is provided which is carried by the central leg 794 of the body 793 and is provided with an o-ring (not shown) which can be moved to close the flow passage in the central leg 794, or alternatively it can be opened to receive a guide wire which can be utilized for advancing the guide catheter 786 if that be necessary and then forming a fluid-tight seal with respect to the guide wire.

Assuming that the guiding catheter 786 has been inserted into the main catheter 651 before insertion of the main catheter 651 into the femoral artery, both catheters can be inserted in unison while advancing the distal extremity of the guide catheter 786 so that it precedes the distal extremity of the main catheter 651 and serves to guide the main catheter 651 into the vessel of interest, as for example the vessel 781 having the stenosis 782 therein. The main catheter 651 is then advanced so that its distal extremity is at the proximal side of the stenosis 782. By way of example, the main catheter 651 can be advanced through the aortic arch of the heart and thence into a saphenous vein graft so that the occlusion balloon 669 on its distal extremity is positioned proximal of the stenosis 782. As soon as this has been accomplished, the guiding catheter 786 can be removed.

As soon as the distal extremity of the main catheter 651 has been deployed so that it is just proximal of the stenosis 782 to be treated, an assembly shown in FIG. 38 is introduced into the main catheter 651. This assembly can be provided by preloading the irrigation catheter 766 onto the therapeutic catheter 801 by inserting the distal tip of the therapeutic catheter 801 through the fitting 769 of the irrigation catheter 766 and advancing the therapeutic catheter 801 until its therapeutic balloon 809 exits from the irrigation catheter 766. The balloon-on-a-wire catheter 701 also is preloaded by removing the valve attachment 746 and then inserting the proximal end 703 into the guide wire lumen at the distal tip of the therapeutic catheter 801 and then advanced proximally until the proximal end protrudes out of the proximal end of the therapeutic catheter. The valve attachment 741 is then reattached to the proximal end 703. The preassembled irrigation catheter 766, the therapeutic catheter 801 and the balloon-on-a-wire catheter 701 are then introduced in unison as an assembly into the main catheter 651. The balloon-on-a-wire device 701 is then advanced until the distal extremity is near the distal extremity of the main catheter 651 but before the distal extremity has been advanced through the stenosis 782.

Let it be assumed that it is now desired to inflate the occlusion balloon 669 carried by the main catheter 651. This can be accomplished in a suitable manner such as with an inflation-deflation device represented schematically by a syringe 802 secured to the fitting 687 (see FIG. 38) and supplying a balloon inflation fluid through the balloon inflation lumen 662 to inflate the occlusion balloon 669 to an occlusion pressure ranging from 1 to 3.9 atmospheres and preferably approximately one to two atmospheres to engage the side wall forming the vessel 781 to occlude the vessel 781 and to prevent further blood flow through the vessel and to thereby provide a working space 803 distal of the occlusion balloon 669. As soon as the occlusion balloon 669 has been inflated, the balloon-on-a-wire device 701 can be advanced across the lesion or stenosis 782 until the deflated occlusion balloon 711 carried thereby is distal of the stenosis 782. It is safe to cross the stenosis 782 because the flow of blood through the stenosis 782 has been occluded by the occlusion balloon 669. Thus if any of the plaque forming the stenosis is dislodged by the occlusion balloon 711 on the balloon-on-a-wire device 701 as the occlusion balloon 711 is crossing the stenosis 782, the plaque particles or emboli 804 will not be carried off by blood. The positive pressure of blood in secondary collaterals or vasculature will prevent emboli from traveling downstream into the secondary vasculature. If desired, aspiration can be supplied to the working space 803 encompassing the stenosis 782 by placing a suitable vacuum connected to the fitting 689 of the main catheter.

The occlusion balloon 711 can then be readily inflated by use of a syringe 805 secured to the fitting 751 of the removable valve fitting or attachment 741 of the balloon-on-a-wire device 701 proximal of the fitting 686 and accessible outside the body of the patient. The occlusion balloon 711 is inflated (see FIG. 39) to at least approximately one to two atmospheres to bracket the stenosis and to determine the size of the working space 803 to provide a chamber. It should be appreciated that the size of this working space or chamber 803 can be adjusted by changing the position of the occlusion balloon 711 in the vessel 781. If desired, this can be accomplished while the occlusion balloon 711 is inflated.

Now let it be assumed that the occlusion balloon 711 has been inflated with the appropriate working space 803 and that it is desired to introduce a therapeutic balloon catheter 801 into the working space 803 to treat the stenosis 782. If the therapeutic catheter 801 is not in the main catheter 651 as hereinbefore described, this can be readily accomplished in the present invention by inserting a plug mandrel 756 into the open end of the lumen 706 of the guide wire 702. After the plug mandrel 756 has been inserted, the syringe 805 can be removed after which the thumb screws 748 and 763 can be loosened to permit the o-rings therein to become decompressed and to release the guide wire 702 and the plug mandrel 756 to permit the fitting or valve attachment 741 to be slipped off to provide a proximal end on the guide wire 702 which is free of obstructions. During removal of the valve attachment or fitting 741, the occlusion balloon 711 remains inflated and continues to be disposed distally of the stenosis 782. The occlusion balloon 669 also remains inflated because the syringe 802 remains attached to the fitting 686 and is disposed proximal of the stenosis 782.

The conventional therapeutic catheter 801 then can be delivered over the guide wire 702 if it is not already present. The therapeutic catheter 801 is provided with a flexible elongate tubular member 806 having proximal and distal extremities 807 and 808 with a central flow passage (not shown) extending between the same. A therapeutic balloon 809 on its distal extremity is adapted to be inflated to therapeutic pressures ranging from 4-20 atmospheres through a balloon inflation lumen (not shown) carried by the flexible elongate tubular member 806 through an adapter 811 mounted on the proximal extremity 807. The therapeutic balloon 809 can be considered to be means for performing work carried by the distal extremity 808 of the flexible elongate tubular member 806. The adapter 811 can be removable of the type hereinbefore described or alternatively can be permanently attached thereto. Assuming that it is a removable adapter, the removable adapter 811 is provided with knobs 812 and 813 carrying o-rings (not shown) adapted to establish fluid-tight seals with the flexible elongate member 806 and the plug mandrel 756, respectively. It is also provided with an inflation port 816 similar to those hereinbefore described which is in communication with the inflation lumen (not shown) provided in the flexible elongate tubular member 806 for inflating the therapeutic balloon 809.

After the balloon catheter 801 has been positioned by the use of radiopaque markers (not shown) conventionally employed in such devices, the therapeutic balloon 809 is disposed so that it is in general alignment with the stenosis 782 as shown in FIG. 39. The therapeutic balloon 809 is then inflated in a conventional manner to perform work by use of an inflation-deflation device schematically represented by the syringe 817 attached to the inflation port 816 to the desired pressure to compress the plaque forming the stenosis 782 as shown in FIG. 40 to increase the size of the opening through the stenosis 782 in the vessel 781.

Let it be assumed that during the compression of the plaque forming the stenosis 782, additional emboli 804 are formed as shown in FIG. 41 by pieces of plaque becoming dislodged from the plaque 782 within the vessel 781. Let it also be assumed that it is desired to remove these emboli before deflation of the occlusion balloons 669 and 711 disposed proximally and distally of the stenosis 782. To accomplish this, the therapeutic balloon 809 is deflated by use of the syringe 817. As soon as this has been accomplished, a saline solution can be introduced through the irrigation catheter 766 by connecting a tube 819 carrying the saline solution from a suitable source as for example a free or pressurized saline bag (not shown) and delivered through the irrigation port or side arm 772 where it is carried through the large central lumen of the irrigation catheter 766 so that the saline solution is discharged into the working space 803 disposed between the occlusion balloons 711 and 669 as shown in FIG. 41. At the same time suitable aspiration means is connected to the aspiration port 689 of the adapter 686 and as shown can consist of a hand operated bulb 821 which has a one way check valve 822 therein connected to the fitting 689.

The bulb 821 is provided with another one-way check valve 823 which is connected to a flexible collection bag 824. The bulb 821 makes it possible to generate a vacuum corresponding approximately to 3-30″ of mercury. Thus, by compressing the bulb 821 by hand, it is possible to create suction within the chamber or space 803 formed in the vessel between the occlusion balloons 669 and 711 each time the bulb 821 is compressed and released. Alternatively, the aspiration can be accomplished by use of a syringe in place of the bulb 821 and the collection bag 824. Saline liquid supplied through the irrigation catheter 766 carrying the emboli 818 is aspirated through the central lumen 657 of the main catheter 651. The aspirated liquid in each cycle of operation created by pressing the bulb 821 is delivered to the collection bag 824. With such a procedure it has been found that it is possible to aspirate emboli as large as 600 μm. Such removal can be assured by observing when clear liquid exits outside the body from the aspiration port 691. A chamber having a length ranging from 3 cm to 15 cm can be totally cleared of emboli within a short period of time ranging from 5 to 30 seconds. Alternatively, irrigation can be accomplished by removing the therapeutic catheter 801 after deflating the therapeutic balloon 809. The irrigation catheter can be advanced over the balloon-on-a-wire device 701 until the distal tip is just proximal of the occlusion balloon 711 as shown in FIG. 42 to provide a greater flow of saline and faster aspiration.

After all of the emboli 804 have been removed, introduction of saline through the tube 819 is halted. It should be appreciated that the ports for irrigation and aspiration can be reversed in function if desired. Thereafter, the occlusion balloon 711 is deflated by removing the plug 756 and utilizing a syringe 805, after which the occlusion balloon 669 is deflated permitting blood flow to be reestablished in the vessel 781. Alternatively, the occlusion balloon 669 can be first deflated and aspiration commenced at that time, permitting emboli trapped distally of the occlusion balloon 669 by blood flowing from the proximal side of the occlusion balloon 669 to be aspirated through the central lumen 657. In order to prevent excessive expansion of the vessel 781 being treated, the pressure of the irrigation liquid is typically maintained under 30 psi. This pressure preferably should be below the occlusion balloon pressure.

If it is desired to deliver a stent to the site of the stenosis formed by the plaque 782, this can be readily accomplished during the same procedure. Typically it is desirable to permit the blood to flow normally for a period of several minutes after which the occlusion balloon 669 can be reinflated by the syringe 805 and the occlusion balloon 711 can be reinflated by inserting the removable valve attachment 741 if it has been removed of the balloon-on-a-wire device 701 and utilizing the syringe 803 to reinflate the occlusion balloon 711. The plug mandrel 756 can be inserted to keep the occlusion balloon 711 inflated after which the valve attachment 741 can be removed.

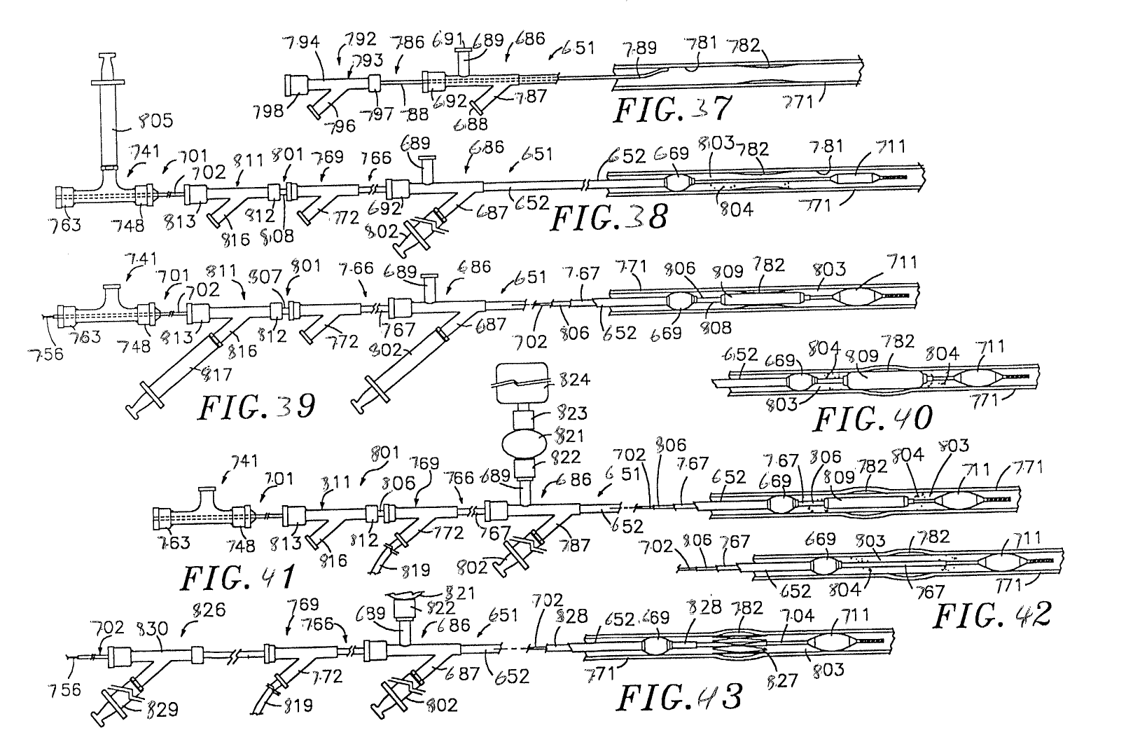

A conventional stent delivery catheter 826 carrying a stent 827 on its flexible shaft 828 is introduced over the balloon-on-a-wire device 701 and delivered to the site of the dilated stenosis 782 (see FIG. 43). The stent 827 can be of the self-expanding type or of the type which can be expanded by a balloon (not shown) carried by the catheter 826 by connecting a syringe 829 to an adapter 830 of the type hereinbefore described of the stent delivery catheter 826. After the stent 827 has been deployed in the dilated stenosis 782, the stent delivery catheter 826 can be removed after which the occlusion balloon 711 can be deflated followed by deflation of the proximal balloon 661 in the manner hereinbefore described. Also it should be appreciated that if desired in connection with the deployment of the stent delivery catheter 826 before it is removed but after deflation of its balloon (not shown), it may be desirable to again flush the working space or chamber 803 between the occlusion balloons 669 and 711 of emboli which may be dislodged during the delivery and deployment of the stent. The irrigation catheter 766 can be deployed in the same manner as hereinbefore described with a saline irrigation solution supplied to the working space 803 in the manner hereinbefore described and liquid aspirated therefrom by the use of the bulb 821 in the manner hereinbefore described.

Heretofore the apparatus of the present invention has been utilized for performing a procedure on a saphenous vein graft where there are no branches to be dealt with. An apparatus incorporating the present invention also can be useful in connection with vessels in a human being having branches therein, as for example the carotids. For this purpose, a main catheter 831 (see FIG. 44) is provided which is very similar to the main catheter 651 with the exception that the adapter 832 provided on the proximal extremity is provided with catheter ports 833 and 834 which are in communication with the large central lumen 657 extending the length of the main catheter. The catheter ports 833 and 834 have a construction similar to the exchange catheter and therapeutic catheter port 692 hereinbefore described in connection with the main catheter 651. These two catheter ports 833 and 834 are necessary because in a carotid procedure, two balloon-on-a-wire devices are utilized. The main catheter should be larger, as for example as large as 12 French, to provide a larger central lumen to accommodate the two balloon-on-a-wire devices.

One of the balloon-on-a-wire devices can be substantially identical to the balloon-on-a-wire device 701 described. The other balloon-on-a-wire device 835 as shown in FIG. 45 differs from the device 701 shown in FIG. 30 in that in place of the removable valve attachment 741 there is provided a fixed adapter 836 which consists of a body 837 provided with diametrically extending wings 838 to facilitate grasping of the adapter 836. The body 837 is provided with a bore 839 which is in communication with the lumen 706 in the guide wire 702. The adapter is provided with a Luer-type fitting 840 to provide a balloon inflation port.

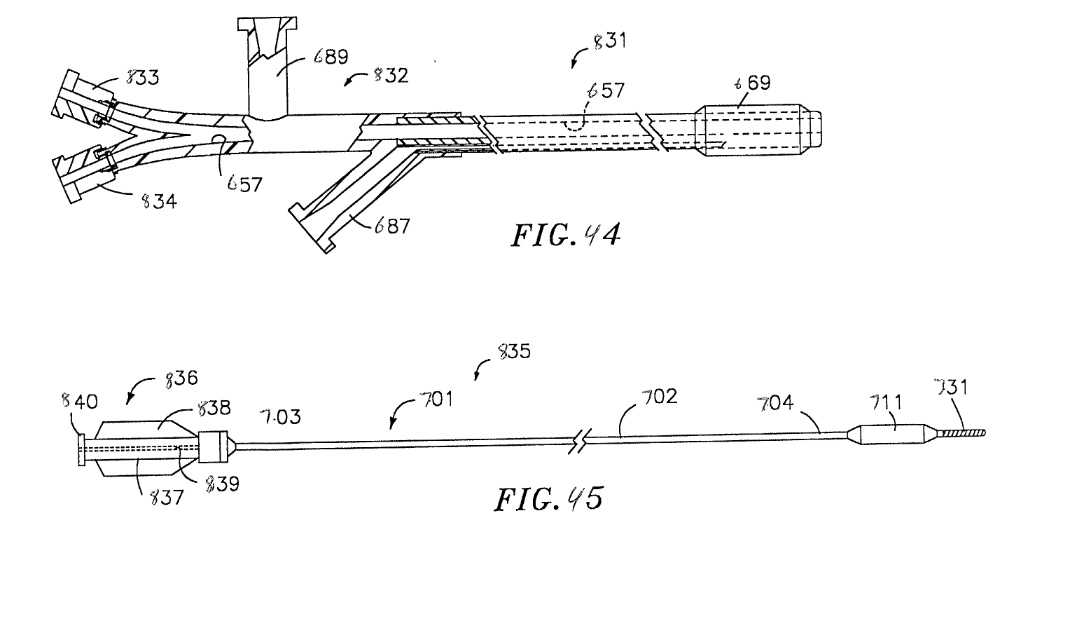

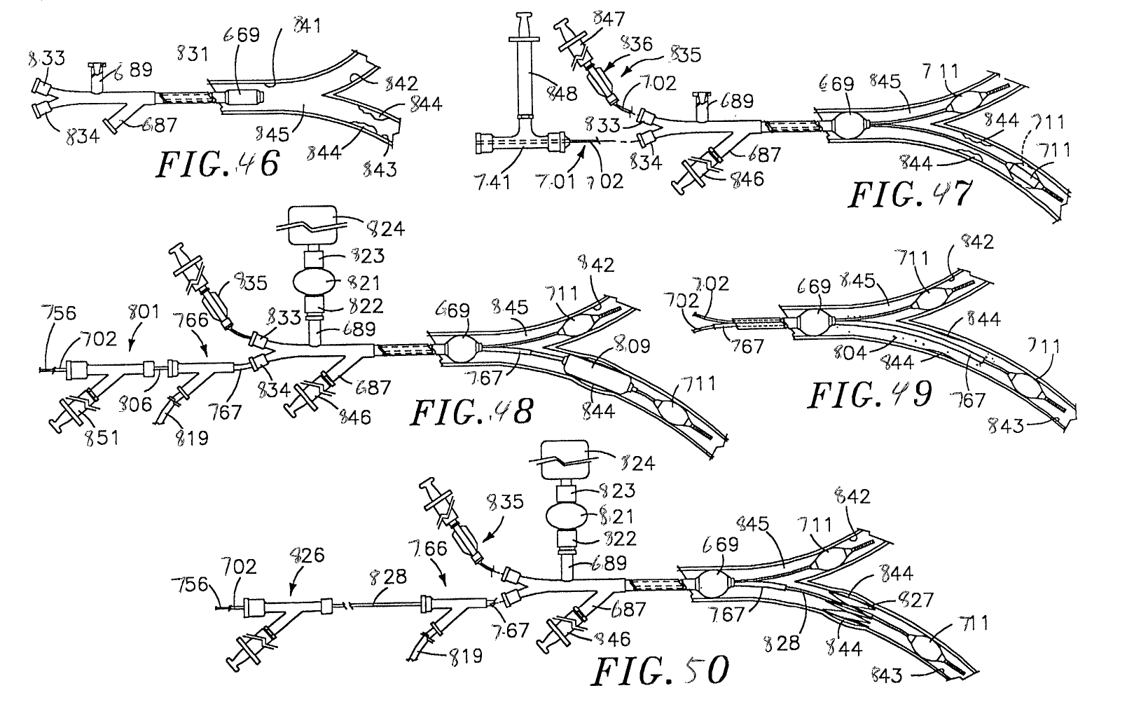

Operation and use of the apparatus of the present invention in performing a procedure in a carotid artery is shown in the cartoons in FIGS. 46-50. Let it be assumed that it is desired to perform a procedure with the apparatus of the present invention on a carotid artery in a patient, as for example common carotid 841 which branches into an external carotid 842 and an internal carotid 843 and that there is a narrowing or a stenosis 844 in the internal carotid 843 near the bifurcation into the external and internal carotids 842 and 843. The main catheter 831 can be introduced in the manner hereinbefore described with respect to a saphenous vein graft. For example it can be introduced through the femoral artery in the leg and then advanced into the aortic arch up into the common carotid 841 until the occlusion balloon 669 carried thereby is near the bifurcation as shown in FIG. 46. The occlusion balloon 669 is then inflated to at least one atmosphere as shown in FIG. 47 to form a seal to occlude the common carotid 841 and to temporarily stop the flow of blood to the face and brain of the patient through the common carotid 841 and to provide a working space 845 distal of the occlusion balloon 669. The inflation is accomplished by suitable means as for example a syringe 846 secured to the balloon inflation fitting 687. Thereafter, a balloon-on-a-wire device 831 of the type shown in FIG. 45 is introduced through the catheter port 833 and advanced through the central lumen 657 of the main catheter 831 after which the distal extremity is guided into the external carotid 842 so it is disposed beyond the bifurcation. The occlusion balloon 711 carried by the distal extremity is then inflated by suitable means such as a syringe 847 secured to the attachment 836 to occlude the external carotid. As hereinbefore pointed out, the balloon 711 is an occlusion balloon that typically is inflated to a suitable occlusion pressure as for example one to two atmospheres.

Another balloon-on-a-wire device such as the balloon-on-a-wire device 701 is then introduced through the catheter port 834 and advanced through the central passage or lumen 657 until it exits from the main catheter 831 after which it is guided into the internal carotid 843 past the stenosis 844 so that the occlusion balloon 711 is distal of the stenosis 844. The occlusion balloon 711 is then inflated as shown by the dotted lines in FIG. 47 by the use of a syringe 848 secured to the inflation port carried by the removable valve attachment 741. Thus, the limits of the working space or chamber 845 are defined by the occlusion balloons 669 and 711. As soon as the balloon 711 has been inflated, the balloon inflation lumen can be plugged in the manner hereinbefore described by the use of a plug mandrel 756 (see FIG. 48). It should be appreciated even though the guide wire 702 and the occlusion balloon 711 carried thereby may dislodge particles from the plaque forming the stenosis 844, the dislodged particles will not travel to the brain because the common carotid supplying blood to the internal carotid 843 has been occluded by the occlusion balloon 669.

The removable valve attachment 741 can then be removed in the manner hereinbefore described so that the proximal extremity of the guide wire 702 is free of obstructions as shown in FIG. 68. Thereafter the irrigation catheter 766 can be introduced over the guide wire 702 and thence into the port 834 until its distal extremity extends beyond the distal extremity of the main catheter 831. A therapeutic balloon catheter 801 of the same type as hereinbefore described can then be introduced through the irrigation catheter 766. It should be appreciated that if desired, the therapeutic balloon catheter can be preloaded into the irrigation catheter 766 and the irrigation catheter 766 and the therapeutic balloon catheter 801 can be introduced in unison. Assuming that the irrigation catheter 766 has been introduced first, the therapeutic balloon catheter 801 is introduced through the irrigation catheter 766 until it extends beyond the distal extremity of the irrigation catheter 766 and is moved into the working space 845 until the therapeutic balloon 809 carried by the distal extremity thereof is in registration with the stenosis 844. The therapeutic balloon 809 is then inflated as shown in FIG. 48 by the use of an inflation/deflation device 851 represented schematically by a syringe to a suitable therapeutic pressure to compress the plaque forming the stenosis 844 to dilate the stenosis to increase the size of the flow passage through the stenosis 844. The therapeutic balloon 809 can then be deflated. In the event emboli 804 are created as hereinbefore described by the passage of the therapeutic balloon 809 through the stenosis, these emboli 804 can be removed as shown in FIG. 49 by introducing a saline solution through the tube 819 and into the irrigation port 773 of the irrigation catheter 766 to cause a saline solution to be discharged into the space formed between the two occlusion balloons 711 and 669. To achieve a more effective aspiration, the distal tip of the irrigation catheter 766 can be moved through the stenosis 844 to just proximal of the occlusion balloon 711. Aspirate is removed through the aspiration port 689 through the use of the bulb 821 and the collection bag 824 to remove the saline solution carrying with it the emboli 804 which may have been created and deposit the same in the collection bag 824. This irrigation and aspiration can be carried on for a suitable period of time as for example 5 to 30 seconds after which the occlusion balloons 711 in both of the branches 842 and 843 can be deflated and the devices 701 and 835 can be removed along with the catheter 801 carrying the therapeutic balloon 809. Similarly, the occlusion balloon 669 can be deflated to permit blood to flow into the common carotid 841 and the external and internal carotids 842 and 843. Alternatively, the sequence of deflation of the balloons can be carried out in the manner hereinbefore described in connection with a vessel without a bifurcation.

In the event it is desired to deliver a stent into the dilated stenosis 844, this can be accomplished by reinflating the occlusion balloon 669 and then reinflating the occlusion balloons 711 in both of the branches after which a balloon stent delivery catheter 826 of the type hereinbefore described can be delivered over the guide wire 702 in the same manner as the therapeutic balloon catheter 766 and delivered into the desired location and then deployed in the dilated stenosis 844. After the stent 827 has been deposited and the balloon of the stent delivery catheter 826 is deflated, the irrigation and aspiration procedures hereinbefore described can be repeated to remove any emboli within the space formed between the occlusion balloons 711 and 669. The stent delivery catheter 826 can be removed. After a suitable period of irrigation and aspiration, as for example 5 to 30 seconds, the occlusion balloon 711 can be deflated after which the occlusion balloon 669 can be deflated and the balloon-on-a-wire devices 701 and 835 removed along with the main catheter 652.

From the foregoing it can be seen that there has been provided a new and improved apparatus and a method for utilization of the same which makes it possible to carry out such stenosis opening procedures without the perfusion of blood. Complete stenosis procedures can be carried out in a period of time which is less than six minutes for each complete procedure. Even though blood flow is occluded during this period of time, this period of time is much less than the period of time, as for example 30 minutes, required for a conventional endoatherectomy. Thus, the procedures of the present invention can be carried out without endangering the patient, as for example the brain or the heart of the patient.

The desire to eliminate the use of a large guiding catheter for use with the main catheter 651 was hereinbefore discussed. Also, it was hereinbefore disclosed that the main catheter 651 can be inserted independently through a conventional sheath (not shown) in the femoral artery and thereafter a smaller conventional guiding catheter 786 is introduced through the main catheter so that its distal extremity 789 is in the vessel. In other procedures it may be desirable to carry this concept still further, ie., eliminating the need for a large guiding catheter and also the need for a smaller guiding catheter to be advanced through the main catheter. To do this, it may be desirable to provide a distal extremity on the main catheter 651 which is shaped in a predetermined manner. For example, in the main catheter 651a shown in FIG. 26A there is provided in the distal extremity a conventional Judkins left shape and in the main catheter 651b shown in FIG. 26B there is provided in the distal extremity a conventional Judkins right shape. Other than the shaping of the distal extremities as hereinbefore described, the main catheters 651a and 651b are constructed in a manner very similar to the catheter 651 and are provided with occlusion balloons 669 as shown.

Since the main catheters 651a and 651b are relatively flexible, they can be inserted into the femoral artery and have their distal extremities guided into the desired locations with the catheter being selected for the appropriate bend to reach the desired location. With the main catheter having such capabilities, it is possible in connection with the present invention to advance the main catheter 651 into the desired location by the use of a balloon-on-a-wire device of the type hereinbefore described, or alternatively over a conventional guide wire. This makes it possible to eliminate the use of a guiding catheter and therefore substantially simplify the procedures of the present invention and reduce the costs of such procedures.

In connection with the irrigation catheter 766 hereinbefore described in FIG. 36, it should be appreciated as shown in FIGS. 36A and 36B that irrigation catheters 766a and 766b can be provided which have soft distal extremities to provide additional flexibility and trackability and thereby reduce trauma in vessels through which they are introduced. Thus in the irrigation catheter 766a shown in FIG. 36A, the main portion of the flexible elongate tubular member 767 which can be considered to be the shaft can have a greater stiffness than the distal portion 767a of the distal extremity. This can be readily accomplished by utilizing a plastic such as Pebax and Hytrel of various desired durometers. For example, the main shaft 767 can have a durometer ranging from 80-100 whereas the distal portion 767a can have a durometer ranging from 50-70. The cylindrical tip 767c with a rounded forward edge as shown is provided with a still lower durometer as for example 35-55 durometer. Thus it can be seen that there has been provided an irrigation catheter which has a very soft tip and which has a distal portion in the distal extremity which is very flexible to permit tracking and to reduce trauma.

In the irrigation catheter 766b shown in FIG. 36, the shaft 767 can have a durometer ranging from 80-100 whereas the portion 767a can have a durometer ranging from 60-70 and which has a portion 767b formed of the same durometer material that is inclined inwardly and distally to reduce the size of the opening for the passage or lumen 768 as shown. The tip 767 which can be formed of a low durometer as for example 35-55 durometer is mounted on the distal extremity 767b. In order to enhance the flow of irrigation fluid from the lumen 768 a plurality of holes 857 is circumferentially distributed around the portion 767a to augment the flow of irrigation fluid other than through the passage 856. The use of the embodiments 766a and 766b of the irrigation catheter is very similar to that hereinbefore described with the irrigation catheter 766 shown in FIG. 36. It should be appreciated that if differing stiffnesses are desired for the main catheters 651 and 831, the same concepts as disclosed for the irrigation catheter 766 can be utilized by selecting materials having desired durometers for various portions of the catheters.

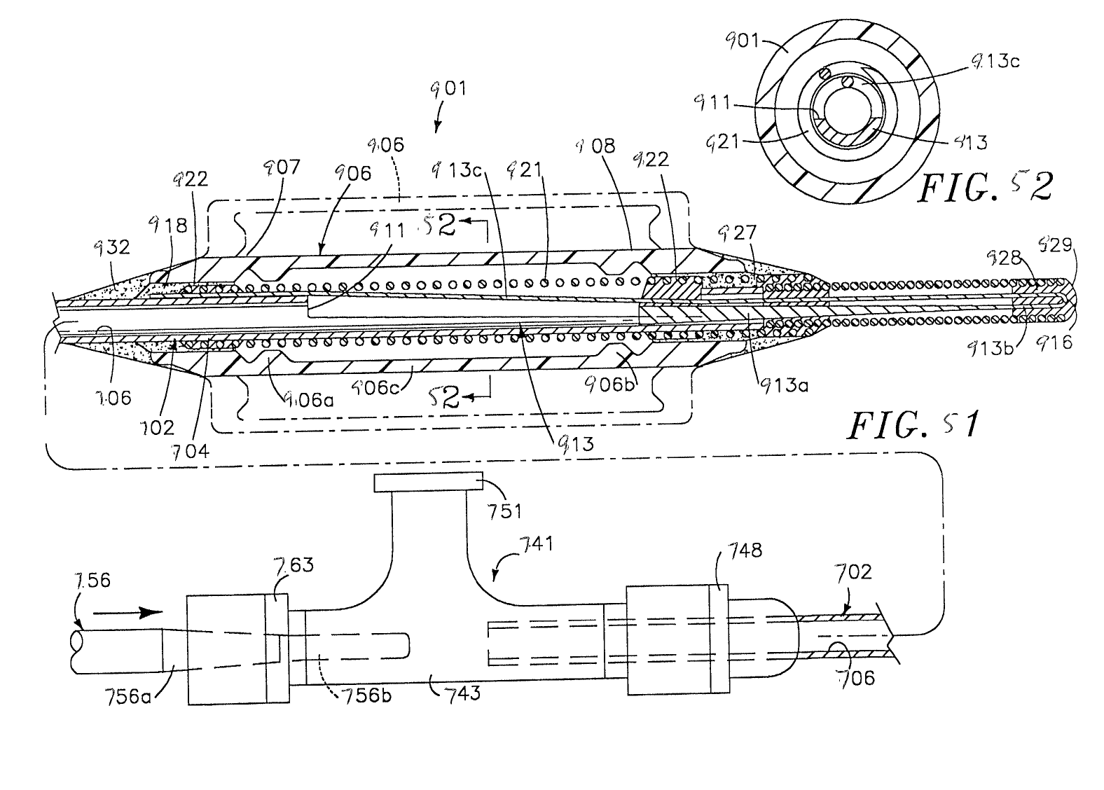

Another embodiment of the balloon-on-a-wire device is shown in FIGS. 51 and 52 in which the balloon-on-a wire device 901 is in many respects very similar to the balloon-on-a-wire device 701 shown in FIG. 30 as hereinbefore described. The balloon-on-a-wire device 901 consists of a flexible elongate member in the form of a guide wire 702 having proximal and distal extremities 703 and 704 with a lumen 706 extending therethrough. A removable valve attachment or fitting 741 is provided on the proximal extremity 907. A plug mandrel 756 is carried by the removable valve attachment 741 for use in plugging the bore 706 when necessary. An elastomeric balloon 906 is provided on the distal extremity 704 and is provided with proximal and distal extremities 907 and 908. The balloon 906 has a suitable length as for example 10 millimeters and a suitable diameter when collapsed or deflated of 1 mm. In order that the balloon 906 assume a generally rectangular shape as viewed in cross-section as shown in FIG. 51 with generally right angle corners, the balloon 906 is provided with spaced-apart cylindrical regions 906a and 906b of greater thickness than an intermediate portion 906c. For example, portions 906a and 906b can have a thickness of 0.006″ to 0.010″ and portion 906c of 0.003″ wall thickness. Such a balloon when inflated will have a squareness as illustrated by the dotted lines in FIG. 51. This squareness of the balloon corners helps to assure that emboli will not become entrapped between the balloon and the vessel wall and thereby will not roll by the balloon as it is moved in the vessel.

An elongate slot 911 is ground into the distal extremity of the guide wire 702 to a suitable depth which is in excess of one half of the diameter of the guide wire 702. The slot 911 is in communication with the lumen 706 and opens into the interior of the balloon 906. A tapered core wire 913 is mounted in the distal extremity 704 of the guide wire 702. The core wire 913 is provided with a portion 913a which has a progressive decrease in diameter extending from the proximal extremity to a portion 913b which is generally of a uniform diameter of a suitable size, as for example 0.003″ and is formed into a bend 916 and extends proximally along the slot 916 and proximally thereof where it is secured to the guide wire 702 by suitable means such as an adhesive 918. A coil spring 921 formed of a suitable material such as stainless steel or platinum extends over the slot 911 and proximally and distally of the slot 911 and is secured thereto by suitable means as solder 922. Positioned in this manner, the coil 921 generally circumscribes the inner circumference of the balloon 906 and serves to protect the balloon 906 from any sharp edges as for example sharp edges formed by the slot 911 in the coil wire 702. A tip coil 926 formed of a suitable radiopaque material such as a platinum or a platinum alloy is mounted over the distal extremity of the guide wire 702 and secured thereto by suitable means such as solder 927. The distal extremity of the tip coil 926 which may have a suitable length, as for example 3 mm, is bonded to the core wire 913b by a solder 928 which encloses the bend 916 and provides a rounded forwardly protruding surface 929. The distal extremity 908 of the balloon 906 is secured to the coils 921 and 926 by an adhesive 931. Similarly, the proximal extremity 907 of the balloon 906 is secured to the guide wire 702 and the portion 913b by an adhesive 932.

The balloon-on-a-wire device 901 can be utilized in the same manner as the balloon-on-a-wire device 701 hereinbefore described. It is believed that the balloon-on-a-wire device 901 has several desirable features. For example the balloon 906 is protected from any sharp edges by the coil spring 921. The slot 911, in addition to providing a means for inflating the balloon, also serves to provide a progressive weakening of the distal extremity of the guide wire 702 to impart additional flexibility to the distal extremity of the device.

By utilizing a balloon-on-a-wire constructions herein disclosed, it is possible to reduce the overall size of the apparatus for the procedures. In view of the fact that guide wires having a size of 0.014″ to 0.018″ are utilized in the present invention, many conventional therapeutic balloon devices can be utilized by advancing the same over such size guide wires. By the provision of removable valve attachments for the balloon-on-a-wire devices, it is possible to use such devices for providing the one or more balloons necessary for a procedure while at the same time making it possible to utilize such devices as guide wires after removing the removable valve attachments on the proximal extremities. This makes it possible to utilize conventional stent delivery catheters, ultrasound catheters and the like by advancing them over the already in place guide wires.

It should be appreciated that it may be possible to eliminate the use of the occlusion balloons 711 which are distal of the proximal balloon carried by the main catheter and distal of the stenosis, since blood flow is occluded during the time that the occlusion balloon 669 is inflated.

Another embodiment of a catheter apparatus incorporating the present invention for treating occluded vessels is shown in FIGS. 53 and 54. As shown therein, the catheter apparatus 951 consists of a flexible elongate member 952 similar to those hereinbefore described which is provided with proximal and distal extremities 953 and 954. A conventional adapter 956 is mounted on the proximal extremity and is provided with a Tuohy-Borst fitting 957 which is in communication with a large central lumen 958 extending from the proximal extremity 953 to the distal extremity 954. An aspiration fitting 961 is provided on the adapter 956 as well as an irrigation fitting 962, both of which are in communication with the central lumen 958. However, it should be appreciated that if desired separate lumens can be provided in the flexible elongate member 952 for both of the fittings 961 and 962.

Self-expanding sealing means 966 is mounted on the distal extremity 954. This self-expanding sealing means 966 can take any suitable form. For example, as shown it can consist of a braided structure 967 formed of a suitable shape memory material such as a nickel titanium alloy that will attempt to expand to a predetermined shape memory. Other than shape memory materials, other materials such as stainless steel, titanium or other materials can be utilized in the braid 967 as long as they have the capability of expanding when the self-expanding seal means is released. Also it should be appreciated that the self-expanding seal means 966 can be comprised of an absorbent material which when it absorbs saline or blood expands to form a seal. Such seals can be readily accomplished because it is only necessary to form a seal of approximately one atmosphere to prevent small particles from moving downstream.

In order to prevent abrasion of a vessel, it is desirable to cover the braided structure 967 with a covering 968 of a suitable material such as a polymer which extends over the braided structure 967 and which moves with the braided structure 967 as it expands and contracts. The polymer can be of a suitable material such as silicone, C-flex, polyethylene or PET which would form a good sealing engagement with the wall of the artery.

Means is provided for compressing the self-expanding sealing means 966 so that the apparatus can be inserted into the vessel 781 and consists of an elongate sleeve 1071 having proximal and distal extremities 1072 and 1073 and a bore 1074 extending from the proximal extremity 1072 to the distal extremity 1073. A collar 1076 is mounted on the proximal extremity 1072 of the sleeve 1071 and is positioned near the adapter 956. The collar 1076 serves as means for retracting the sleeve as shown in FIG. 54 to uncover the self-expanding sealing means 966 after the catheter has been deployed to permit the self-expanding sealing means 966 to expand and form a seal with the arterial vessel adjacent the stenosis to be treated.

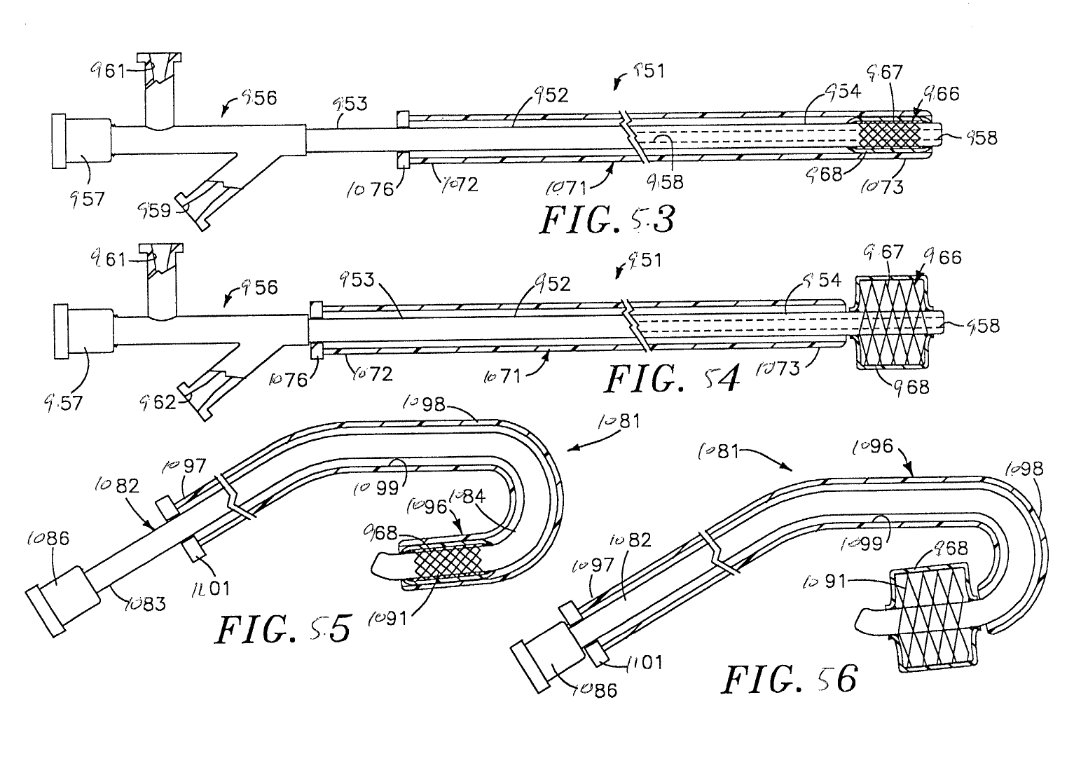

Another embodiment of a catheter apparatus for treating occluded vessels incorporating the present invention is shown in FIGS. 55 and 56. As shown therein, the apparatus 1081 consists of a guiding catheter 1082 having proximal and distal extremities 1083 and 1084. As shown, the distal extremity 1083 is provided with a pre-formed bend of a conventional type. A conventional attachment 1086 is mounted on the proximal extremity 1083. Self-expanding seal means 1091 is mounted on the distal extremity 1084 and is of the type hereinbefore described in connection with the embodiments shown in FIGS. 53 and 54. A sleeve 1096 similar to the sleeve 1071 of the previous embodiment is provided in the present embodiment for encasing the self-expanding seal means 1091 and for releasing the same after it has been disposed in an appropriate position within a vessel adjacent the occlusion to be treated. Thus a sleeve 1096 is provided having proximal and distal extremities 1097 and 1098 and having a bore 1099 extending from the proximal extremity to the distal extremity which is sized so that it can receive the guide catheter 1082. It is provided with a collar 1101 on its proximal extremity which is adapted to be disposed outside the patient and which is adapted to be grasped by the physician for pulling the sleeve 1096 proximally to uncover the self-expanding seal 1091 after the apparatus has been deployed to permit the self-expansion of the sealing means 1091 to form a seal with the vessel wall is shown in FIG. 56.

In accordance with the hereinbefore described descriptions, it is apparent that the apparatus can be readily deployed and serve the same function as the main catheter. To accomplish this, the assembly 1081 can be introduced into the femoral artery and the distal extremity advanced into the desired location in the arterial vessel. After it has been properly positioned, the physician can retract the sleeve 1096 to permit the self-expanding seal means 1091 to expand and to form a seal with the wall of the arterial vessel to occlude the arterial vessel and interrupt the flow of blood in the vessel to provide a working space distal of the occlusion formed. This prevents small particles which may thereafter be dislodged from moving downstream. Since a central lumen is available, the therapeutic procedures hereinbefore described can be employed with the catheter apparatus shown in FIGS. 53, 54, 55 and 56.

Thus it can be seen that it has been possible to substantially reduce the complexity of the apparatus utilized in such procedures. This reduces the cost of the apparatus used therein as well as reducing the time required for performing such procedures making the procedures less costly.

It will be appreciated that certain variations of the present invention may suggest themselves to those skilled in the art. The foregoing detailed description is to be clearly understood as given by way of illustration, the spirit and scope of this invention being limited solely by the appended claims.

What is claimed is:

1. A guiding catheter for positioning a medical device within an artery, said guiding catheter comprising:
a flexible elongate member having an outer surface, a distal region adapted to enter an artery, a proximal region extending outside the artery, a port in the distal region, and a lumen extending from the proximal region to the distal region, and communicating with the port, wherein the lumen is adapted to receive a medical device which can pass through the port to access the lumen of the artery;
an expandable filter disposed within the distal region of the elongate member and proximal the port, the filter being expandable between a contracted condition and an enlarged condition, the filter having a first proximal edge circumferentially in contact with the outer surface of the flexible elongate member, wherein during use plaque disrupted by the medical device is captured by the filter.

2. The guiding catheter of claim 1, wherein a distal segment of the flexible elongate member extends beyond the expandable filter and is bent at an angle of greater than 10° relative to a segment proximal the distal segment so as to direct a medical device toward the vessel wall.

3. A guiding catheter for positioning a medical device within an artery, said guiding catheter comprising:
a flexible elongate member having an outer surface, a distal region adapted to enter an artery, a proximal region extending outside the artery, a port in the distal region, and a lumen extending from the proximal region to the distal region and communicating with the port, wherein the lumen is adapted to receive a medical device which can pass through the port to access the lumen of the artery; and
at least one positioning member disposed within the distal region of the flexible elongate member and proximal the port, wherein during use said positioning member is deployed to stabilize or deflect the elongate member within the vessel lumen.

4. The guiding catheter of claim 3, wherein a distal segment of the flexible elongate member extends beyond the at least one positioning member and is bent at an angle of greater than 10° relative to a segment proximal the distal segment so as to direct a medical device toward the vessel wall.

5. The guiding catheter of claim 3, wherein the at least one positioning member comprises a mechanical extending arm operable from the proximal region of the elongate member.

6. The guiding catheter of claim 3, wherein the at least one positioning member comprises an balloon which is inflatable by a separate lumen within the guiding catheter.

7. The guiding catheter of claim 6, wherein the at least one positioning member comprises an elastomeric balloon.

8. The guiding catheter of claim 3, further comprising an expandable filter disposed within the distal region of the flexible elongate member.

9. The guiding catheter of claim 8, wherein the expandable filter is downstream of the at least one positioning member.

10. A method for protecting a patient from embolization during a percutaneous medical procedure, comprising the steps of:
providing a guiding catheter comprising a flexible elongate member having a lumen extending from a proximal to a distal region of the elongate member, and having an expandable filter within the distal region of the elongate member;
advancing the distal region of the guiding catheter to a region of interest within a vessel of the patient;
expanding the expandable filter downstream of the region of interest;
deploying a medical instrument through the lumen of the elongate member; and
performing a percutaneous procedure on the vessel upstream of the filter, wherein embolic material dislodged by the procedure is captured by the filter.

11. The method of claim 10, wherein the guiding catheter further includes at least one positioning member within the distal region of the elongate member, and wherein the method further comprises the step of deploying said at least one positioning member to deflect or stabilize the guiding catheter within the vessel.

12. The method of claim 11, wherein said at least one positioning member is upstream of the expandable filter.

13. The method of claim 10, wherein the medical instrument is an atherectomy device.
